(19) United States
(12) Patent Application Publication
(10) Pub. No.: US 2022/0040018 A1
(43) Pub. Date: Feb. 10, 2022

(54) Smart Bed or Mattress for Reducing Pressure Ulcers Which Uses Adjustable Orthogonal Straps to Create Low-Pressure Areas

(71) Applicant: Robert A. Connor, St. Paul, MN (US)

(72) Inventor: Robert A. Connor, St. Paul, MN (US)

(73) Assignee: Sleepnea LLC, St. Paul, MN (US)

(21) Appl. No.: 17/511,210

(22) Filed: Oct. 26, 2021

Related U.S. Application Data
(63) Continuation-in-part of application No. 16/826,197, filed on Mar. 21, 2020.
(60) Provisional application No. 62/830,497, filed on Apr. 7, 2019.

(51) Int. Cl. A61G 7/057; A61B 5/00

(52) U.S. Cl. CPC A61G 7/057; A61B 2562/0247; A61G 2203/34; A61B 5/6892

(57) ABSTRACT

This invention is a smart bed or mattress for reducing pressure ulcers with an orthogonal array of movable straps or layers which creates a low-pressure area. The location of the low-pressure area is selected to be under a portion of a person's body which is at risk for getting a pressure ulcer. A low-pressure area can be created: where straps which have been slackened overlap; where elastic segments of movable straps overlap; or where the movement of straps creates an opening.

### CROSS-REFERENCE TO RELATED APPLICATIONS

This application is a continuation-in-part of U.S. patent application Ser. No. 16/826,197 filed on 2020 Mar. 21. U.S. patent application Ser. No. 16/826,197 claimed the priority benefit of U.S. provisional patent application 62/830,497 filed on 2019 Apr. 7. The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to devices for reducing pressure ulcers on people in bed.

### Introduction

Pressure ulcers (also known as decubitus ulcers or bed sores) are localized areas of tissue damage and/or necrosis which are caused by prolonged pressure on the same tissue location, sheer friction on the tissue, localized high moisture level, incontinence or high perspiration, high temperature, poor blood circulation, anemia, infection, poor nutrition, inactivity or immobility, sensory impairment, and/or advanced age. Pressure ulcers tend to form where there is prolonged external pressure on tissue at a bony prominence such as the sacrum, coccyx, hips, heels, ankles, knees, and elbows.

Pressure ulcers can result in pain and suffering, open wounds, infection and sepsis, disability, amputation, and even death. The prevalence of pressure ulcers among people in intensive care units and nursing homes continues to be high despite ongoing efforts toward prevention, with high associated costs in terms of human suffering as well as direct healthcare costs. There remains a significant need for development of better devices and methods to help prevent pressure ulcers, especially with the aging population. This need is increasing due to the increase in the elderly population, economic constraints on human labor to turn patients from one side to the other, and the increase in contagious diseases. The devices and methods disclosed herein provide some innovative ways to help prevent and heal decubitus ulcers.

### Review of the Prior Art

U.S. Pat. No. 5,010,608 (Barnett et al., Apr. 30, 1991, “Support System for Reducing Formation of Decubitus Ulcers”) discloses a system with cells that can be individually inflated and deflated. U.S. patent application 20180311095 (Bednarz et al., Nov. 1, 2018, “Pressure Redistribution System and Methods of Using Same”) discloses a chair with a frame, a back cushion inflation system, and a plurality of back cushion air bladders. U.S. patent Ser. No. 10/667,973 (Bidoki, Jun. 2, 2020, “Multi-Purpose System for a Hospital Bed”) discloses a bed with vertically-movable lifting members that are parallel and spaced-apart elongated parts transversally connected to the bed. U.S. Pat. No. 9,763,841 (Carlson et al., Sep. 19, 2017, “Apparatus and Method for Automatic Adjustment of a Support Surface with Interwoven Support Elements”) and U.S. patent application 20140103687 (Carlson et al., Apr. 17, 2014, “Apparatus and Method for Automatic Adjustment of a Support Surface with Interwoven Support Elements”) disclose an apparatus for adjusting the lengths of a plurality of interwoven support elements to conform to contours of a user's anatomy.

U.S. patent application 20170128297 (Cernasov et al., May 11, 2017, “Supporting Surface with Programmable Supports and Method to Reduce Pressure on Selected Areas of a Body”) and U.S. patent Ser. No. 10/531,996 (Cernasov et al., Jan. 14, 2020, “Supporting Surface with Programmable Supports and Method to Reduce Pressure on Selected Areas of a Body”) disclose a body support device with at least one sensor and an array of programmable supports.

U.S. patent Ser. No. 10/045,632 (Chapin, Aug. 14, 2018, “Traveling Wave Air Mattresses and Method and Apparatus for Generating Traveling Waves Thereon”), U.S. Pat. No. 9,015,885 (Chapin, Apr. 28, 2015, “Traveling Wave Air Mattresses and Method and Apparatus for Generating Traveling Waves Thereon”), U.S. Pat. No. 9,888,784 (Chapin, Feb. 13, 2018, “Traveling Wave Air Mattresses and Method and Apparatus for Generating Traveling Waves Thereon”), and U.S. patent application 20180160821 (Chapin, Jun. 14, 2018, “Traveling Wave Air Mattresses and Method and Apparatus for Generating Traveling Waves Thereon”) disclose a traveling wave air mattress apparatus with an array of laterally disposed, longitudinally spaced air bladder cells.

U.S. patent application 20120068510 (Chen, Mar. 22, 2012, “Pressure-Ulcer-Prevention Dynamic Cushion”) and U.S. Pat. No. 8,719,980 (Chen, May 13, 2014, “Pressure-Ulcer-Prevention Dynamic Cushion”) disclose a multiple-phase dynamic cushion with clusters of strips interleaved in parallel. U.S. Pat. No. 9,724,257 (Chen et al., Aug. 8, 2017, “Anti-Bedsore Bed”) discloses a bed unit with six supporting units arranged at positions corresponding to a user's upper, middle and lower back. U.S. patent application 20110190674 (Dahl et al., Aug. 4, 2011, “Support Device for the Establishment of a Controlled Flow of Blood in a Body Part”) discloses a piston grid which adjusts support pressure and aid blood flow. U.S. Pat. No. 9,149,401 (Davenport et al., Oct. 6, 2015, “Support Apparatus for Preventing and/or Inhibiting Decubitus Ulcers”) discloses an apparatus with a pair of frame supports which are spaced from each other.

U.S. Pat. No. 8,898,842 (Dennis, Dec. 2, 2014, “Anti-Decubitus Ulcer Mattress Overlay System with Selective Elevation Structure”) and U.S. Pat. No. 9,693,916 (Dennis, Jul. 4, 2017, “Anti-Decubitus Ulcer Mattress Overlay System with Selective Elevation Structure”) an elevation structure system with multiple elongate, positionally-adjustable, relatively-moveable risers. U.S. patent application 20160008206 (Devanaboyina, Jan. 14, 2016, “Systems and Methods for Exerting Force on Bodies”) discloses a support structure with a plurality of surface-contacting units. U.S. patent application 20160317370 (Evans et al., Nov. 3, 2016, “Cushion with Bladders Running Different Pressurization Modes Inside and Outside Dynamically Selected Target Bladder Group”) discloses a responsive cushion system with a pressure adjustment system. U.S. patent 20040221390 (Frey, Nov. 11, 2004, “Bed Including User Adaptable Support Structure”) discloses a support structure with a frame and a plurality of flexible support straps anchored between side members of the frame.

U.S. Pat. No. 7,905,846 (Ganti, Mar. 15, 2011, “Special Bed, for Bedsores Therapy and Massage Therapy”) discloses a bed with moving parallel lateral rods. U.S. patent application 20180280219 (Garrett et al., Oct. 4, 2018, “Air Mattress Turning Device”) discloses an apparatus comprising first and second sets of bladders for rotating a bed-ridden person. U.S. patent application 20150128354 (Greenstein et al., May 14, 2015, “Methods, Circuits, Apparatuses and Assemblies for Providing a Mattress or Body Portion Support Cushion with a Sensor Layer”) discloses a resting body support comprising a sensor layer in a mattress adjustment system. U.S. Pat. No. 5,283,735 (Gross et al., Feb. 1, 1994, “Feedback System for Load Bearing Surface”) discloses a chair or bed an array of pressure sensors.

U.S. Pat. No. 5,963,997 (Hagopian, Oct. 12, 1999, “Low Air Loss Patient Support System Providing Active Feedback Pressure Sensing and Correction Capabilities for Use as a Bed Mattress and a Wheelchair Seating System”) discloses a therapeutic air support system with real time pressure adjustments. U.S. patent Ser. No. 10/039,488 (Hermann et al., Aug. 7, 2018, “System and Method to Determine Tissue Compression”) discloses a system to measure body tissue compression in a non-contact manner. U.S. patent application 20100094175 (Hovorka, Apr. 15, 2010, “Adjustable Pneumatic Supporting Surface”), U.S. Pat. No. 8,801,635 (Hovorka, Aug. 12, 2014, “Adjustable Pneumatic Supporting Surface”), U.S. patent application 20140350434 (Hovorka, Nov. 27, 2014, “Adjustable Pneumatic Supporting Surface”), and U.S. Pat. No. 9,730,585 (Hovorka, Aug. 15, 2017, “Adjustable Pneumatic Supporting Surface”) disclose an apparatus with a plurality of inflatable bladders which apply kinetic energy to human tissue.

U.S. patent applications 20130019408 (Jacofsky et al., Jan. 24, 2013, “Systems and Methods for Monitoring and Providing Therapeutic Support for a User”) and 20160256100 (Jacofsky et al., Sep. 8, 2016, “Systems and Methods for Monitoring and Providing Therapeutic Support for a User”) disclose a method for monitoring pressure and automatically adjusting one or more inflatable sections of a support structure. U.S. Pat. No. 7,467,426 (Jarmon et al., Dec. 23, 2008, “Body Support and Method for Supporting a Body”) discloses a body support with a first and a second plurality of interdigitated flexible parallel bands. U.S. Pat. No. 4,799,276 (Kadish, Jan. 24, 1989, “Body Rest with Means for Preventing Pressure Sores”) discloses a bed with a matrix of vertical displaceable supports. U.S. patent application 20100268121 (Kilborn, Oct. 21, 2010, “Active Support Surface”) discloses a support structure with subframes and electroactive polymer actuators.

U.S. patent application 20160324707 (Lachenbruch et al., Nov. 10, 2016, “Multi-Mode Sacral Unloading Pressure Relief in a Patient Support Surface”) discloses a mattress including a support bladder system and a rotation bladder system. U.S. patent applications 20110263950 (Larson et al., Oct. 27, 2011, “Systems, Devices and Methods for Preventing, Detecting and Treating Pressure-Induced Ischemia, Pressure Ulcers, and Other Conditions”), 20160278691 (Larson et al., Sep. 29, 2016, “Systems, Devices and Methods for Preventing, Detecting, and Treating Pressure-Induced Ischemia, Pressure Ulcers, and Other Conditions”), and 20160278692 (Larson et al., Sep. 29, 2016, “Systems, Devices and Methods for Preventing, Detecting, and Treating Pressure-Induced Ischemia, Pressure Ulcers, and Other Conditions”) disclose a support surface with sensors therein and/or sensors affixed to a patient.

U.S. patent application 20130281804 (Lee et al., Oct. 24, 2013, “Methods and Systems for Monitoring a Patient to Reduce the Incidence of Pressure Ulcers”) discloses monitoring a patient using a sensor array disposed between the patient and a support surface. U.S. Pat. No. 8,893,338 (McCausland et al., Nov. 25, 2014, “Inflatable Cellular Mattress with Alternating Zones of Inflated Cells”) discloses a cushion or mattress with a plurality of linearly-aligned air cells. U.S. patent application 20130090571 (Nourani et al., Apr. 11, 2013, “Methods and Systems for Monitoring and Preventing Pressure Ulcers”) a pressure mapping system that records a patient's bed posture.

U.S. Pat. No. 5,659,905 (Palmer et al., Aug. 26, 1997, “Patient Transfer/Turning Bed”) discloses a bed with rotating drive rollers and articulating arms which turn a person. U.S. Pat. No. 8,752,222 (Papaioannou, Jun. 17, 2014, “Adaptable Surface for Use in Beds and Chairs to Reduce Occurrence of Pressure Ulcers”), patent application 20100101026 (Papaioannou, Apr. 29, 2010, “Adaptable Surface for Use in Beds and Chairs to Reduce Occurrence of Pressure Ulcers”) and patent application 20150128352 (Papaioannou, May 14, 2015, “Adaptable Surface for Use in Beds and Chairs to Reduce Occurrence of Pressure Ulcers”) disclose an adaptable surface system with a plurality of individually adjustable surface units and a plurality of pressure sensors.

U.S. patent application 20170348181 (Perriard et al., Dec. 7, 2017, “System for Adjusting Pressure Locally on the Skin and Subcutaneous Tissue”) discloses a system for adjusting pressure with a set of adjacent modules capable of changing shape. U.S. patent Ser. No. 10/058,189 (Petrov, Aug. 28, 2018, “Active Multicompartmental Pressure Redistribution System”) and patent application 20160037939 (Petrov, Feb. 11, 2016, “Active Multicompartmental Pressure Redistribution System”) disclose a multicompartmental pressure redistribution system that identifies contact body pressure points and redistributes pressure in real time. U.S. Pat. No. 7,515,059 (Price et al., Apr. 7, 2009, “Patient Support Surface with Physiological Sensors”) discloses a support surface with an air bladder and a sensor to detect a physiological parameter of a patient on the surface. U.S. Pat. No. 9,295,600 (Receveur, Mar. 29, 2016, “Person Support Apparatus with Activity and Mobility Sensing”) discloses a person support apparatus with sensors to monitor changes in a person's position.

U.S. patent application 20160125716 (Ribble et al., May 5, 2016, “Equipment, Dressing and Garment Wireless Connectivity to a Patient Bed”), U.S. Pat. No. 9,711,029 (Ribble et al., Jul. 18, 2017, “Equipment, Dressing and Garment Wireless Connectivity to a Patient Bed”), U.S. patent Ser. No. 10/037,674 (Ribble et al., Jul. 31, 2018, “Equipment, Dressing, and Garment Wireless Connectivity to a Patient Bed”), and U.S. patent application 20170270766 (Ribble et al., Sep. 21, 2017, “Equipment, Dressing, and Garment Wireless Connectivity to a Patient Bed”) disclose a patient bed having a reader to read wireless signals, such as those from a wound dressing. U.S. patent application 20190029903 (Riley et al., Jan. 31, 2019, “Person Support Apparatus Having Physiological Sensor”) discloses a system with a frame, a support surface, and a sensor.

U.S. Pat. No. 8,407,833 (Sauter et al., Apr. 2, 2013, “Deformable Supporting Element and Reclining System”) discloses a deformable support element for use in a bed system. U.S. patent application 20180369040 (Sheth, Dec. 27, 2018, “System and Method for Treating and Preventing Pressure Sores in Bedridden Patients”) and U.S. patent application 20180369041 (Sheth, Dec. 27, 2018, “System and Method for Treating and Preventing Pressure Sores in Bedridden Patients”) disclose a system with an array of expandable and collapsible supports to provide pressure relief to a patient. U.S. Pat. No. 5,815,864 (Sloop, Oct. 6, 1998, “Microprocessor Controller and Method of Initializing and Controlling Low Air Loss Floatation Mattress”) discloses a pressurization and control system for an air floatation mattress or cushion. U.S. Pat. No. 9,504,620 (Soltani et al., Nov. 29, 2016, “Method of Controlling a Pressurized Mattress System for a Support Structure”) and U.S. patent application 20160022519 (Soltani et al., Jan. 28, 2016, “Method of Controlling a Pressurized Mattress System for a Support Structure”) disclose a method for automatically varying the pressure in an air bladder in a zone of a pressurized mattress.

U.S. Pat. No. 8,726,908 (Squitieri, May 20, 2014, “Enhanced Patient-Orienting Alternating Pressure Support Apparatus”), U.S. Pat. No. 9,931,238 (Squitieri, Apr. 3, 2018, “Enhanced Patient-Orienting Alternating Pressure Support Apparatus”), U.S. patent application 20140290670 (Squitieri, Oct. 2, 2014, “Enhanced Patient-Orienting Alternating Pressure Support Apparatus”), U.S. Pat. No. 8,757,165 (Squitieri, Jun. 24, 2014, “Patient-Orienting Alternating Pressure Support Apparatus with Lower Extremity Wedge”), U.S. Pat. No. 9,901,491 (Squitieri, Feb. 27, 2018, “Patient-Orienting Alternating Pressure Support Apparatus with Lower Extremity Wedge”), U.S. patent application 20150164677 (Squitieri, Jun. 18, 2015, “Patient-Orienting Alternating Pressure Support Apparatus with Lower Extremity Wedge”), and U.S. patent application 20180185205 (Squitieri, Jul. 5, 2018, “Patient-Orienting Alternating Pressure Support Apparatus with Lower Extremity Wedge”) disclose systems to mitigate pressure by actively orienting a patient over an anatomy-specific pressure-mitigating contact surface.

U.S. patent application 20090194115 (Squitieri, Aug. 6, 2009, “Apparatus for Prevention and Treatment of Decubitus Ulcers”) discloses a garment with a bladder to prevent and/or treat pressure ulcers. U.S. patent application 20110263950 (Larson et al., Oct. 27, 2011, “Systems, Devices and Methods for Preventing, Detecting and Treating Pressure-Induced Ischemia, Pressure Ulcers, and Other Conditions”) discloses a system with sensors for monitoring pressure ulcers. U.S. patent application 20190021918 (Squitieri, Jan. 24, 2019, “Non-Invasive Pressure-Mitigation Apparatuses for Improving Blood Flow and Associated Systems and Methods”) discloses a pressure-mitigation apparatus with a series of chambers whose pressure can be individually varied.

U.S. patent application 20160228050 (Sugla et al., Aug. 11, 2016, “System and Method for Prevention of Pressure Ulcers”) discloses a system including a pressure sensor attached to a patient to prevent pressure ulcers. U.S. Pat. No. 8,813,284 (Teasdale et al., Aug. 26, 2014, “Inflatable Component for an Alternating Pressure Mattress”) discloses an inflatable mattress with at least two inflatable cells. U.S. Pat. No. 8,539,627 (Terawaki et al., Sep. 24, 2013, “Body Position and Pressure Control Apparatus”) discloses a body position and pressure control apparatus for detecting body pressure distribution. U.S. Pat. No. 5,052,067 (Thomas et al., Oct. 1, 1991, “Bimodal System for Pressurizing a Low Air Loss Patient Support”) discloses a patient support system with a plurality of inflatable sacks. U.S. Pat. No. 7,823,232 (Tinke et al., Nov. 2, 2010, “Device for Preventing Decubitus”) discloses a device for preventing decubitus with adjacent members which are covered with a layer of elastic material and connected to one another via a pulling element.

U.S. Pat. No. 5,267,364 (Volk, Dec. 7, 1993, “Therapeutic Wave Mattress”) discloses a mattress with a plurality of inflatable tubular elements. U.S. patent application 20180310719 (Wiggermann, Nov. 1, 2018, “Adaptable Mattress”) discloses a mattress with a phase changeable component and a thermal management system. U.S. Pat. No. 6,560,804 (Wise et al., May 13, 2003, “System and Methods for Mattress Control in Relation to Patient Distance”) discloses a system for monitoring the distance between a patient and an inflatable air mattress and for controlling the air supply. U.S. patent application 20180061044 (Woodbridge et al., Mar. 1, 2018, “Method and System for Monitoring a User to Prevent Pressure Ulcers”) discloses monitoring users to prevent pressure ulcers using imaging technologies to detect the user's posture. U.S. Pat. No. 5,794,289 (Wortman et al., Aug. 18, 1998, “Mattress for Relieving Pressure Ulcers”) discloses a mattress with a fabric strip bridging a foam crib to provide lateral stability when the mattress is tilted to turn a patient.

### SUMMARY OF THIS INVENTION

Disclosed herein is a smart bed or mattress for reducing pressure ulcers. This smart bed or mattress has an orthogonal array of movable straps or layers. These straps or layers are selectively and independently moved relative to each other in order to create one or more low-pressure areas on the upper surface of the bed or mattress. The one or more low-pressure areas are positioned under one or more portions of a person's body which are at risk for getting a pressure ulcer or already have a pressure ulcer. A low-pressure area reduces the pressure exerted by the bed or mattress on a vulnerable portion of the person's body, thereby helping to prevent (or heal) a pressure ulcer.

In an example, a low-pressure area is created at a selected location where straps which have been slackened overlap. In an example, a low-pressure area is created at a selected location where elastic segments of movable straps overlap. In an example, a low-pressure area is created at a selected location where the movement of straps creates an opening. In an example, a low-pressure area is created at a selected location where an opening in an upper layer and an opening in a lower layer are aligned.

The smart bed or mattress disclosed herein has potential advantages over the prior art. Compared to beds or mattresses in the prior art with a uniform pressure level across an entire bed or mattress surface, the smart bed or mattress disclosed herein enables the creation of low-pressure areas at one or more selected locations under a portions of a person's body which are particularly at risk for getting pressure ulcers. Also, the disclosed smart bed or mattress enables controlled movement of the locations of a low-pressure area over time, which decreases the chances of a pressure ulcer due to prolonged pressure on a particular portion of a person's body.

Compared to beds or mattresses in the prior art with a grid of individual inflation-adjustable air-filled (or fluid-filled) cells, the smart bed or mattress disclosed herein reduces the accumulation of moisture and heat which are harmful for skin health. Individually-adjustable cells filled with air or fluid have surfaces which are impermeable to air. This reduces airflow to a person's body, which can lead to moist and warm conditions which cause pressure ulcers. The air-permeable design of the disclosed smart bed or mattress can avoid this problem.

Compared to beds or mattresses in the prior art with a grid of individual mechanical height-adjustable cells, the smart bed or mattress disclosed herein achieves substantially the same result, but with greatly-reduced mechanical complexity and a more-comfortable sleeping surface for the resting person. For example, if one wishes to have 100 mechanical individually-adjustable cells in a bed or mattress using designs in the prior art, one would need 100 individual mechanical cells, with the attendant mechanical complexity of making and controlling that many cells. Individual pistons or solenoids can be used for such mechanical height-adjustable cells, but are likely to create uncomfortable bumps for the resting person as well as being mechanically complex. In contrast, the design disclosed herein can selectively create low-pressure areas in 100 different locations by adjustment of 20 overlapping straps, 10 of which span the bed or mattress in a first direction and 10 of which span the bed or mattress in a second direction. Also, the disclosed design is less likely to create uncomfortable bumps.

For these reasons, the smart bed or mattress disclosed herein has the potential to prevent pressure ulcers more effectively, more comfortably, and at lower cost than beds or mattresses in the prior art.

### BRIEF INTRODUCTION TO THE FIGURES

FIGS. 1 and 2 show an example of a smart bed or mattress for reducing pressure ulcers with an orthogonal array of overlapping straps which are selectively slackened to create a low-pressure area.

FIGS. 3 and 4 show an example of a smart bed or mattress for reducing pressure ulcers with an orthogonal array of overlapping straps with elastic segments which are selectively aligned to create a low-pressure area.

FIGS. 5 and 6 show an example of a smart bed or mattress for reducing pressure ulcers with an orthogonal array of straps which are selectively moved to create an opening which serves as a low-pressure area.

FIGS. 7 and 8 show an example of a smart bed or mattress for reducing pressure ulcers with upper and lower layers with openings which are moved and selectively aligned to create a low-pressure area.

### DETAILED DESCRIPTION OF THE FIGURES

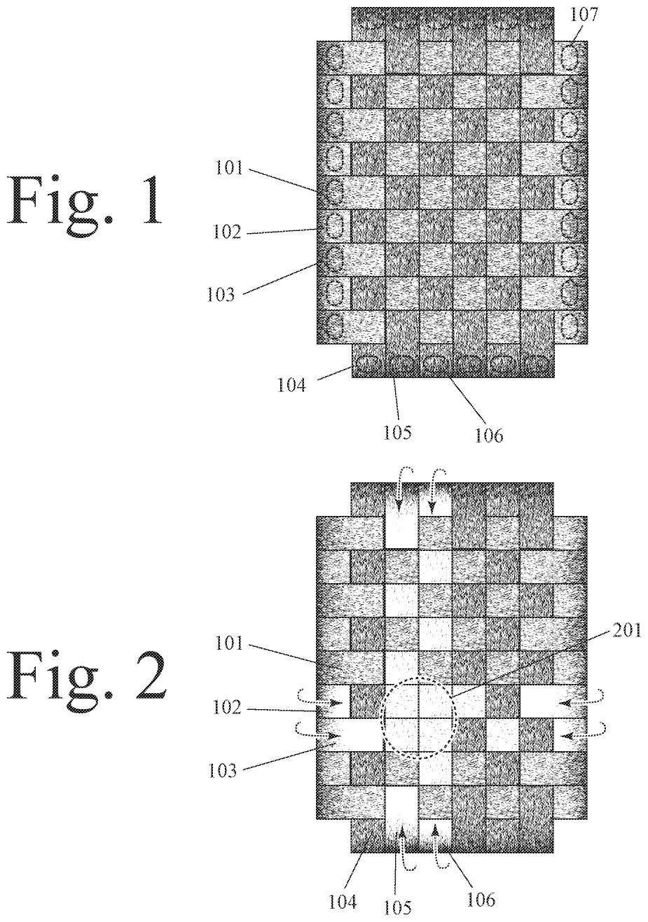

FIGS. 1 and 2 show two top-down views, at two different times, of an example of a smart bed or mattress for reducing pressure ulcers. FIG. 1 shows this smart bed or mattress at a first point in time, before selected straps in an orthogonal array of straps have been selectively and automatically loosened to create a low-pressure area on the bed or mattress. FIG. 2 shows this smart bed or mattress at a second point in time, after selected straps in the orthogonal array of straps have been selectively and automatically loosened to create a low-pressure area on the bed or mattress. A low-pressure area is created where loosened straps spanning different directions across the bed or mattress overlap in the orthogonal array. The size and location of the low-pressure area is selected so as to reduce pressure on a portion of a person's body which is at high risk of getting a pressure ulcer or which already has a pressure ulcer.

The smart bed or mattress for reducing pressure ulcers which is shown in FIGS. 1 and 2 comprises: (a) a bed or mattress on which a person rests; (b) at least one electromagnetic motor (only shown in FIG. 1); (c) a first set of flexible straps which span the bed or mattress in a head-to-foot direction; wherein a first subset of straps comprises one or more straps selected from the first set; wherein straps in the first subset have a taut configuration with a first length spanning the bed or mattress and a slack configuration with a second length spanning the bed or mattress; wherein the second length is greater than the first length; wherein straps in the first subset are moved from the taut configurations to the slack configurations, or vice versa, by one or more motors selected from the at least one motor; wherein different first subsets are selected at different times to create low-pressure areas with different sizes and/or locations on the bed or mattress; and wherein the selection of straps in the first subset is based at least partly on predetermined timing, a position of the person's body, analysis of camera images, data from infrared sensors, data from stretch sensors, and/or data from pressure sensors; and (d) a second set of flexible straps which span the bed or mattress in a right-to-left direction; wherein a second subset of straps comprises one or more straps selected from the second set; wherein straps in the second subset have a taut configuration with a third length spanning the bed or mattress and a slack configuration with a fourth length spanning the bed or mattress; wherein the fourth length is greater than the third length; wherein straps in the second subset are moved from the taut configurations to the slack configurations, or vice versa, by one or more motors selected from the at least one motor; wherein different second subsets are selected at different times to create low-pressure areas with different sizes and/or locations on the bed or mattress; wherein the selection of straps in the second subset is based at least partly on predetermined timing, a position of the person's body, analysis of camera images, data from infrared sensors, data from stretch sensors, and/or data from pressure sensors; wherein a low-pressure area is an area on the bed or mattress where pressure exerted by the bed or mattress on the person's body is reduced relative to other areas of the bed or mattress; and wherein a low-pressure area is formed where the first and second subsets overlap when straps in the first and second subsets are in their slack configurations.

With respect to specific components, the smart bed or mattress shown in FIGS. 1 and 2 comprises: a plurality of electromagnetic motors (including 107); a first set of flexible straps (including 104, 105, and 106) which span the bed or mattress in a head-to-foot direction; wherein a first subset of straps (105 and 106) from the first set have a taut configuration (see FIG. 1) with a first length spanning the bed or mattress and a slack configuration (see FIG. 2) with a second length spanning the bed or mattress; wherein second length is greater than the first length; wherein straps in the first subset are moved from the taut configurations to the slack configurations; and a second set of flexible straps (including 101, 102, and 103) which span the bed or mattress in a right-to-left direction; wherein a second subset of straps (102 and 103) selected from the second set have a taut configuration (see FIG. 1) with a third length spanning the bed or mattress and a slack configuration (see FIG. 2) with a fourth length spanning the bed or mattress; wherein fourth length is greater than the third length; wherein straps in the second subset are moved from the taut configurations to the slack configurations; wherein a low-pressure area 201 is an area on the bed or mattress where pressure exerted by the bed or mattress on the person's body is reduced relative to other areas of the bed or mattress; and wherein the low-pressure area is formed where the first and second subsets overlap when straps in the first and second subsets are in their slack configurations. The motors are not shown in FIG. 2 to reduce diagrammatic clutter.

In an example, straps can span the upper surface of a bed or mattress. In an example, straps can be close to the upper surface of a bed or mattress, but there can be a sheet (or other flexible layer) above the straps, between the straps and a person's body. In an example, a sheet (or other flexible layer) between the straps and a person's body can protect the person from abrasion when the straps move and protect them from being pinched by gaps between straps. In an example, straps or a sheet (or other flexible layer) can further comprise an array of pressure sensors whose data is analyzed to measure area-specific pressure levels of a person's body on a strap matrix. In an example, the device can further comprise a lift mechanism which temporarily lifts a person up before the straps move and lowers the person down after the straps have moved.

In an example, this device can comprise a smart bed or smart mattress. In an example, this device can comprise a smart mattress pad, smart sheet, or smart blanket. In an example, this device can comprise a flexible net, mesh, sling, or hammock. In an example, this device can be a stand-alone product. In an example, this device can be used in combination with an existing bed or mattress. In an example, this device can be used on top of an existing bed or mattress. In an example, straps can be made from a strong, but also flexible and breathable, fabric. In variations on this example, strips, bands, belts, loops, or cords can be used instead of straps.

In an example, straps in a first set of straps can be parallel to each other. In an example, straps in a second set of straps can be parallel to each other. In an example, straps in a second set of straps can be orthogonal to straps in a first set of straps, forming quadrilateral (e.g. square-shaped) overlap areas. In an example, straps in a second set of straps can be interwoven with straps in a first set of straps. Alternatively, straps in a second set of straps can uniformly overlay straps in a first set of straps. In a variation on this example, there can be three sets of straps, wherein straps in different sets intersect and/or overlap each other at 60-degree angles, forming six-sided (e.g. hexagonal-shaped) overlap areas. In an example, straps in first and second sets can be woven together to create an orthogonal mesh or grid which supports a sleeping person, wherein a selected area of the grid can be changed into a low-pressure area by changing the lengths (e.g. level of slack or tension) of straps which overlap at that area.

In an example, a low-pressure area can be formed from a contiguous combination of overlap areas. In the example shown in FIGS. 1 and 2, a square low-pressure area is formed from a contiguous combination of four square overlap areas. In an example, a first subset of straps and a second subset of straps each comprise multiple straps in their slack configurations. In the example shown in FIGS. 1 and 2, the first subset of straps comprises two straps and the second subset of straps comprises two straps. In an example, a low-pressure area can have a quadrilateral shape. In an example, a quadrilateral low-pressure area can have a first side comprising multiple strap widths and a second side comprising multiple strap widths. In an example, the size and/or location of a low-pressure area on a bed can be selected so that it is under a portion of a person's body which has a pressure ulcer or is at risk for getting a pressure ulcer. In this manner, that portion of the person's body will have less force exerted on it from the bed surface.

In an example, straps can be between 2 inches and 2 feet wide. In an example, a first set of straps which spans a bed in a head-to-foot direction can comprise at least 4 straps. In an example, a first set of straps which spans a bed in a head-to-foot direction can comprise between 3 and 10 straps. In an example, a second set of straps which spans a bed in a right-to-left direction can comprise at least 5 straps. In an example, a second set of straps which spans a bed in a right-to-left direction can comprise between 4 and 15 straps. In an example, a first set of straps which spans a bed in a head-to-foot direction can comprise at least 4 straps, a second set of straps which spans a bed in a right-to-left direction can comprise at least 5 straps, and there can be at least (4×5=) 20 areas of overlap between the first and second sets wherein low-pressure areas can be selectively formed. In an example, a first set of straps which spans a bed in a head-to-foot direction can comprise at least 10 straps, a second set of straps which spans a bed in a right-to-left direction can comprise at least 10 straps, and there can be at least (10×10=) 100 areas of overlap between the first and second sets wherein low-pressure areas can be selectively formed.

In an example, the size and/or location of a low-pressure area on a bed can be changed over time to avoid prolonged pressure on a particular portion of a person's body. In an example, the location of a low-pressure area can be moved like a wave across the surface of a bed. In an example, the location of a low-pressure area can be moved across the surface of a bed according to a programmed pattern. In an example, the location of a low-pressure area can be moved across the surface of a bed according to a programmed pattern which includes movement in a head-to-toe direction and movement in a right-to-left direction.

In an example, a low-pressure area can have a concave shape relative to the rest of the upper surface of a bed. In an example, an area of overlap between two perpendicular straps which are both slack will form a concavity when it comes into contact with a portion of a person's body resting on it. This concavity provides less resistance and thus exerts less pressure on that portion of the person's body. In an example, areas of overlap wherein one of the overlapping straps is taut and the other overlapping strap is slack will be less concave, or not concave at all, due to support from the taut trap. However, areas of overlap wherein both of the overlapping straps are slack will be concave.

In an example, a low-pressure area which is created by this device can offer less resistance to a person's body than other areas of the surface. In an example, a low-pressure area which is created by this device can have greater elasticity than other areas of the surface. In an example, a low-pressure area which is created by this device can be more stretchable than other areas of the surface. In an example, a low-pressure area which is created by this device on the surface of a bed or mattress can have a lower durometer level than other areas of the surface. In an example, a low-pressure area which is created by this device on the surface of a bed or mattress can be an opening or hole formed by openings (or holes) through two otherwise-overlapping straps.

In an example, this device can comprise at least one electromagnetic motor (and/or actuator) which moves one or more straps. In an example, at least one electromagnetic motor can be incorporated into a bed or mattress. In an example, at least one electromagnetic motor can be located inside a bed or mattress. In this example, there are a plurality of electromagnetic motors which move straps. In this example, there is a first set of motors which move (e.g. adjust the length and tension of) straps in the first set of straps which spans a bed in a head-to-toe direction and a second set of motors which move (e.g. adjust the length and tension of) straps in the second set of straps which spans a bed in a right-to-left direction. In this example, a motors individually adjust the lengths and/or tensions (e.g. slack vs. taut) of straps.

In this example, there are two electromagnetic motors for each strap, one on each end of a strap. In an example, these electromagnetic motors can control the rotation of spools which wind, or unwind, each end of each strap. As the strap unwinds from a spool, the portion of the strap spanning the surface of the bed or mattress becomes longer and more slack. As the strap winds around the spool, the portion of the strap spanning the surface of the bed or mattress becomes shorter and more taut. Alternatively, there may be only one electromagnetic motor per strap; there may be a spool on only one end of a strap which winds, or unwinds, the strap. The motors and spools can be located inside the bed or mattress for injury reduction and a less-cluttered appearance. The straps can roll around rollers along the sides of the bed or mattress and enter the interior of the bed or mattress through a thin slot to engage with the motors and spools.

Alternatively, an electromagnetic motor can control a piston which changes the length and/or tension of a strap. A strap can be connected to the moving end of a piston. Alternatively, an electromagnetic motor can control a rotating threaded mechanism which changes the length and/or tension of a strap. As the threaded mechanism turns, it loosens or tightens a connected strap. Alternatively, an electromagnetic motor can control a gear mechanism which changes the length and/or tension of a strap. Alternatively, one electromagnetic motor can selectively move a plurality of straps via a set of mechanical gears or linkages. Alternatively, a strap can pass between two rollers which are moved by a motor, wherein rotation of the rollers moves the strap. Alternatively, a motor can move a strap with an elastic or thin segment laterally across a bed or mattress, thereby moving the location of the elastic or thin segment relative to the bed or mattress.

In an example, flexible straps can be individually and independently moved between their taut and slack configurations. In an example, flexible straps in first and second sets can be analogous to a matrix with columns and rows, wherein a particular cell in the matrix can be activated by activating a selected column and row. When a selected head-to-toe strap is made slack and a selected right-to-left strap is made slack, the area where they overlap also becomes a low-pressure area. In an example, this device can further comprising a data processor which receives input data from human commands and/or sensors and selects which motors to activate to create a low-pressure area at a desired location based on this input data. For example, as shown in FIGS. 1 and 2, a data processor may activate motors to slacken straps 102, 103, 105, and 106 in order to create a low-pressure area 201 because this area is below a bony protrusion of a resting person's body (as detected by a camera or pressure sensors) which is at risk for getting a pressure ulcer. In an example, this device can further comprise a wireless data transmitter by which the data processor is in wireless communication with a remote device such as a cell phone.

In an example, a device for reducing pressure ulcers by creating one or more low-pressure areas on a support surface on which a person rests can have three layers: an upper layer (such as a sheet) which is continuous, breathable, and flexible; a middle layer which is a woven orthogonal matrix of straps whose levels of slack can be individually and independently changed; and a lower level which is air space or a highly-compressible foam. In an example, a device for reducing pressure ulcers by creating one or more low-pressure areas on a support surface on which a person rests can have four layers: an upper layer (such as a sheet) which is continuous, breathable, and flexible; a first middle layer comprising a first set of flexible parallel straps which spans a bed in a first direction, wherein the slack levels of a subset of straps in this first set can be adjusted; a second middle layer comprising a second set of flexible parallel straps which spans the bed in a second direction which is orthogonal to the first direction, wherein the slack levels of a subset of straps in this second set can be adjusted; and a lower level which is air space or a highly-compressible foam.

This device with rows and columns of straps can enable adjustment of low-pressure areas of different sizes and/or locations across the surface of a bed with much less mechanical complexity than a device with a separate adjustment mechanism (such as an inflatable cell or individual piston cell) for each area. For example, with 10 adjustment mechanisms to adjust 10 rows (e.g. 10 straps which span a bed in a right-to-left direction) and 10 mechanisms to adjust 10 columns (e.g. 10 straps which span the bed in a head-to-foot direction), one can selectively and individually adjust the pressure of 100 different cells (overlap areas) on the bed. This only takes 20 adjustment mechanisms. In contrast, with a device with one adjustment mechanism for each cell, it would take 100 adjustment mechanisms. This is why most beds in the prior art with a matrix of individually-inflatable cells become mechanically complex very quickly. The device disclosed herein offers less mechanical complexity than devices with individual inflatable cells. The bed disclosed herein also has a more air-permeable surface which is better for skin health than a bed with inflatable cells which can retain moisture and heat.

In an example, the straps can be loops. In an example, straps which span the upper surface of a bed or mattress can loop around the sides of the bed or mattress and engage with one or more slack-adjustment mechanisms (e.g. one or more motors) which are below the upper surface of the bed or mattress. In an example, straps can loop around rollers on the sides of the bed. In an example, when the upper portions of rollers on both side of a bed both rotate toward the center of a bed, then a strap between these rollers becomes more slack (and less taut). In an example, when the upper portions of rollers on both side of a bed both rotate away the center of a bed, then a strap between these rollers becomes more taut (and less slack). In example, each strap can have its own roller on each side of the bed so that each strap can be moved independently. In an example, a roller can be a passive (non-mechanized) roller which reduces friction as a strap which loops around the side of a bed is moved by a slack-adjustment mechanism (e.g. a motor) below the surface of the bed or mattress. Alternative, a roller can be an active roller (e.g. directly moved by a motor) which actively engages and moves a strap.

In an example, one or more ends of a strap can wind around one or more spools. In an example, when such a spool turns in a first direction and unwinds the strap, then the portion of the strap spanning the upper portion of the bed or mattress becomes longer, more slack, less taut, and less tense. In an example, when such a spool turns in the other direction and winds the strap, then the portion of the strap spanning the upper portion of the bed or mattress becomes shorter, more taut, less slack, and more tense. In an example, spools at the ends of a strap can be within the interior of a bed or mattress, controlling the length, degree of slack, and degree of tension in the portion of the strap which spans the upper surface of the bed or mattress.

In a variation on this example, there can be longitudinal variation in the thickness of straps. In other words, different segments along the length of a strap can have different thicknesses. Thin segments can be overlapped to form low-pressure areas. In a variation on this example, there can be longitudinal variation in the elasticity and/or flexibility of straps. In other words, different segments along the length of a strap can have different levels of elasticity and/or flexibility. Elastic segments can be overlapped to form low-pressure areas. In a variation on this example, there can be longitudinal variation in the width of straps. In other words, different segments along the length of a strap can have different widths. In a variation on this example, there can be one or more openings and/or holes at one or more locations along the length of a strap. In an example, lateral movement of one or more selected straps can change the thickness, elasticity, flexibility, width, and/or resistance of the upper surface of a bed or mattress in one or more selected areas. In an example, a device can further comprise a stationary hollow sheath for each strap, wherein a sheath contains a strap which is moved. The moving strap changes the elasticity and/or concavity of an area to create a low-pressure area. The sheath around the moving strap prevents the moving strap from abrading the person's body or pulling a bottom sheet during its movement.

In an example, a strap can be a loop, wherein an upper portion of the loop spans the upper surface of a bed or mattress and a lower portion of the loop spans an interior portion of the bed or mattress. In an example, rollers can engage the loop and rotate it so that portions of the loop switch places, wherein portions which were on the surface now move inside the bed or mattress and portions which were inside the bed or mattress now move to the surface. In this manner, portions of the strap with greater elasticity can be moved to, or from, the surface of a bed or mattress and to different locations on the surface of the bed or mattress. In an example, this device can further comprise a drying and/or cleaning mechanism which dries and/or cleans a portion of a loop as that portion passes through the interior of a bed or mattress (e.g. through a section of the bed or mattress other than its upper surface).

In an example, this device can be part of a system which automatically adjusts the size and/or location one or more low-pressure areas on a person's bed based on a predetermined pattern and/or schedule. In an example, this device can be part of a system which periodically varies the sizes and/or locations of one or more low-pressure areas on a person's bed in order to reduced prolonged periods of high pressure for any particular portion of the person's body. In an example, adjacent areas of a bed or mattress can alternate over time between a normal-pressure configuration and a low-pressure configuration.

In an example, this device can be part of a system which adjusts the size and/or location of one or more low-pressure areas on a person's bed or mattress based on commands from a human-to-computer interface. In an example, these commands can be voice commands from the person or a caregiver. In an example, these commands can be touch-based (e.g. button or touch-screen) commands from the resting person or from a caregiver. In an example, such a system can adjust the size and/or location of one or more low-pressure areas in real time in response to feedback from the resting person (e.g. with respect to comfort). In an example, such a system can adjust the size and/or location of one or more low-pressure areas in response to documented progress in the healing of pressure ulcers (e.g. reinforcing pressure patterns associated with healing and reducing patterns associated with pathology).

In an example, this device can be part of a system which automatically adjusts the size and/or location one or more low-pressure areas on a person's bed or mattress based on data from one or more sensors. In an example, these sensors can indicated the location of a portion of the person's body which is at risk for a pressure ulcer and a low-pressure area can be created underneath than portion in order to prevent a pressure ulcer. In an example, this device can be part of a system which automatically adjusts the size and/or location a low-pressure area on a person's bed based on data from sensors regarding the location of a portion of the person's body with a pressure ulcer in order to help heal the pressure ulcer. In an example, these one or more sensors can be selected from the group consisting of: a camera which records images in the visible spectrum; an infrared camera and/or sensor; a plurality of electromagnetic (e.g. resistive or capacitive) stretch or bend sensors which are integrated into the bed, mattress, or a sheet; a plurality of pressure sensors on the upper surface of a bed or mattress; a plurality of pressure sensors on a garment worn by the person; and a garment with electromagnetic energy emitters worn by the person whose signals are used to identify the person's body configuration.

In an example, this device can further comprise a video camera whose images are analyzed to recognize a person's body configuration on a bed or mattress and thereby identify the locations of one or more portions of the person's body which at risk for pressure ulcers. In an example, one or more low-pressure areas can be created under those one or more portions. In an example, this device can further comprise an infrared imager whose data is analyzed to recognize a person's body configuration on a bed or mattress and thereby identify the locations of one or more portions of the person's body which at risk for pressure ulcers. In an example, one or more low-pressure areas can be created under those one or more portions.

In an example, this device can further comprise a plurality (e.g. an array, grid, or matrix) of pressure sensors whose data is analyzed to recognize a person's body configuration on a bed or mattress and thereby identify the locations of one or more portions of the person's body which at risk for pressure ulcers. In an example, one or more low-pressure areas can be created under those one or more portions. In an example, this device can further comprise a plurality (e.g. an array, grid, or matrix) of stretch and/or bend sensors whose data is analyzed to recognize a person's body configuration on a bed or mattress and thereby identify the locations of one or more portions of the person's body which at risk for pressure ulcers. In an example, one or more low-pressure areas can be created under those one or more portions. Example variations discussed elsewhere in this disclosure or in priority-linked disclosures can also be applied to this example where relevant.

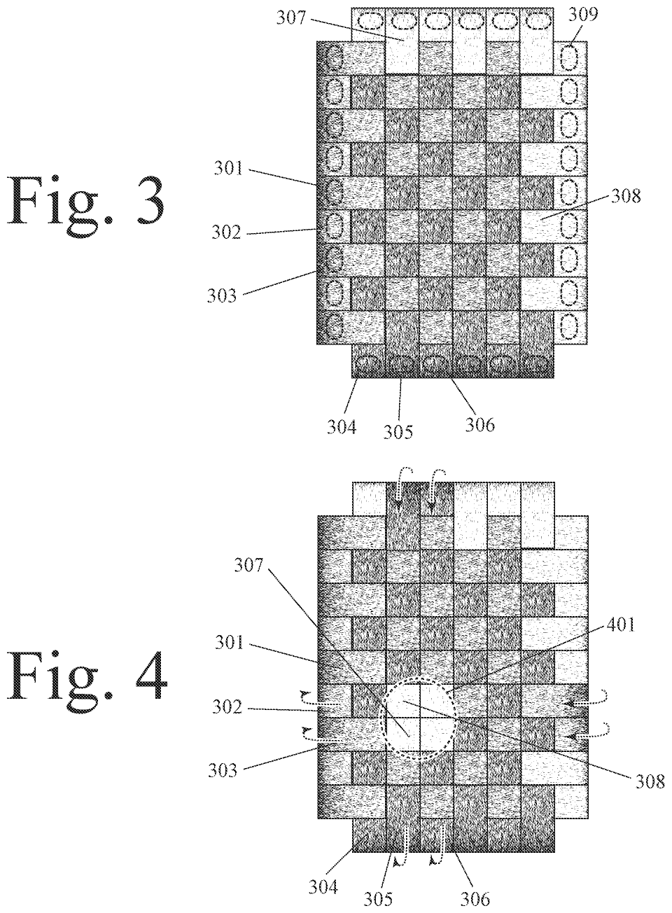

FIGS. 3 and 4 show two top-down views, at two different times, of an example of a smart bed or mattress for reducing pressure ulcers. This example is similar to the one just shown in FIGS. 1 and 2 in that it also has an orthogonal array of straps which are adjusted to create a localized low-pressure area on a bed or mattress. However, unlike the example shown in FIGS. 1 and 2, straps in this present example have elastic (or thin) segments which are moved to overlap in order to create a low-pressure area. In this example, selected subsets of straps are moved laterally (e.g. shifted), rather than being lengthened or shortened, in order to create a low-pressure area in a desired location.

FIG. 3 shows this smart bed or mattress at a first point in time, before selected straps in an orthogonal array of straps have been selectively and automatically moved to align elastic (or thin) segments to create a low-pressure area on the bed or mattress. FIG. 4 shows this smart bed or mattress at a second point in time, after selected straps in the orthogonal array of straps have been selectively and automatically moved to align elastic (or thin) segments to create a low-pressure area on the bed or mattress. A low-pressure area is created where elastic (or thin) segments of straps spanning different directions overlap in the orthogonal array. The size and location of the low-pressure area is selected so as to reduce pressure on a portion of a person's body which is at high risk of getting a pressure ulcer or which already has a pressure ulcer.

The smart bed or mattress for reducing pressure ulcers which is shown in FIGS. 3 and 4 comprises: (a) a bed or mattress on which a person rests; (b) at least one electromagnetic motor (only shown in FIG. 3); (c) a first set of flexible straps which span the bed or mattress in a head-to-foot direction; a first subset of straps comprising one or more straps selected from the first set; wherein each strap in the first subset has an elastic (or thin) segment which is more elastic (or thinner) than the rest of the strap; wherein each strap in the first subset is moved (e.g. shifted) by one or more motors so that the elastic (or thin) segment is moved to a selected location where a low-pressure area is desired; and wherein the selected location is based at least partly on predetermined timing, a position of the person's body, analysis of camera images, data from infrared sensors, data from stretch sensors, and/or data from pressure sensors; and (d) a second set of flexible straps which span the bed or mattress in a right-to-left direction; a second subset of straps comprising one or more straps selected from the second set; wherein each strap in the second subset has an elastic (or thin) segment which is more elastic (or thinner) than the rest of the strap; wherein each strap in the second subset is moved (e.g. shifted) by one or more motors so that the elastic (or thin) segment is moved to a selected location where a low-pressure area is desired; wherein the selected location is based at least partly on predetermined timing, a position of the person's body, analysis of camera images, data from infrared sensors, data from stretch sensors, and/or data from pressure sensors; wherein a low-pressure area is an area on the bed or mattress where pressure exerted by the bed or mattress on the person's body is reduced relative to other areas of the bed or mattress; and wherein a low-pressure area is formed where the first and second subsets overlap after their elastic (or thin) segments have been moved.

With respect to specific components, the device shown in FIGS. 3 and 4 comprises: a plurality of electromagnetic motors (including 309); a first set of flexible straps (including 304, 305, and 306) which span a bed or mattress in a head-to-foot direction, a first subset of straps (305 and 306) which are selected from the first set; a second set of flexible straps (including 301, 302, and 303) which span the bed or mattress in a right-to-left direction, and a second subset of straps (302 and 303) which are selected from the second set. FIGS. 3 and 4 also show elastic (or thin) segments (including 307) of the first subset of straps and elastic (or thin) segments (including 308) of the second subset of straps. In FIG. 3, these elastic (or thin) segments are all along the sides of the bed or mattress. Alternatively, they could be inside the bed or mattress. In FIG. 4, elastic (or thin) segments of the first and second subsets have been moved to overlap at a selected interior location, which creates low-pressure area 401 at that location. The motors are not shown in FIG. 4 in order to reduce diagrammatic clutter.

In an example, movable straps can span the upper surface of a bed or mattress. In an example, these straps can be close to the upper surface of a bed or mattress, but there can be a sheet (or other flexible layer) between the straps and a resting person's body. A sheet or other flexible layer between the straps and a person's body can protect the person from abrasion when the straps move and from being pinched by gaps between straps. In an example, there can be an array (e.g. a grid or matrix) of pressure sensors on this sheet (or other flexible layer) or on the straps themselves. In an example, data from the pressure sensors can be analyzed to measure area-specific pressure levels of a person's body on the surface of a bed or mattress and determine where to create one or more low-pressure areas. In an example, a device can further comprise a lift mechanism which temporarily lifts a person up before straps are moved and lowers the person after the straps have been moved.

In an example, this device can comprise a smart bed or smart mattress. In an example, this device can comprise a smart mattress pad, smart sheet, or smart blanket. In an example, this device can comprise a flexible net, mesh, sling, or hammock. In an example, this device can be a stand-alone bed or mattress product. In an example, this device can be used in combination with an existing bed or mattress. In an example, this device can be used on top of an existing bed or mattress. In an example, straps can be made from a breathable fabric. In a variation on this example, strips, bands, belts, loops, or cords can be used instead of straps.

In an example, straps in a first set of straps can be parallel to each other. In an example, straps in a second set of straps can be parallel to each other. In an example, straps in a second set of straps can be orthogonal to straps in a first set of straps, forming quadrilateral (e.g. square-shaped) overlap areas. In an example, straps in a second set of straps can be interwoven with straps in a first set of straps. In an example, straps in a second set of straps can overlay straps in a first set of straps. In a variation on this example, there can be three sets of straps, wherein straps in different sets intersect and/or overlap each other at 60-degree angles, forming six-sided (e.g. hexagonal-shaped) overlap areas. In an example, straps in first and second sets can be woven together to create an orthogonal mesh or grid which supports a sleeping person, wherein a selected area of the grid can be changed into a low-pressure area by aligning the elastic segments of the straps which overlap at that area.

In an example straps can be between 2 inches and 2 feet wide. In an example, a first set of straps which spans a bed in a head-to-foot direction can comprise at least 4 straps. In an example, a second set of straps which spans a bed in a right-to-left direction can comprise at least 5 straps. In an example, a first set of straps which spans a bed in a head-to-foot direction can comprise at least 4 straps, a second set of straps which spans a bed in a right-to-left direction can comprise at least 5 straps, and there can be at least (4×5=) 20 areas of overlap between the first and second sets wherein low-pressure areas can be selectively formed.

In an example, a first segment of a strap can have a first elasticity level and a second segment of the strap can have a second elasticity level, wherein the second elasticity level is greater than the first elasticity level. In an example, a low-pressure area can be created by aligning the high elasticity segments of orthogonal overlapping straps. A low-pressure area occurs where there are overlapping high elasticity segments in straps in both directions. In an example, a first segment of a strap can have a first thickness and a second segment of the strap can have a second thickness, wherein the second thickness is less than the first thickness. In an example, a low-pressure area can be created by aligning the thin segments of orthogonal overlapping straps. A low-pressure area occurs where there are overlapping thin segments in straps in both directions.

In an example, the length of an elastic (or thin) segment of a strap can be equal to the width of the strap. In an example, the length of an elastic (or thin) segment of a strap can be 1-2 times the width of the strap. In an example, there can be two or more elastic (or thin) segments on a strap, which enables the creation of two or more low-pressure discontinuous areas on the surface of a bed or mattress. In an example, a low-pressure area is formed from a contiguous combination of overlapping elastic (or thin) segments. In an example, a low-pressure area can have a quadrilateral shape. In an example, a quadrilateral low-pressure area can have a first side comprising multiple strap widths and a second side comprising multiple strap widths. In an example, the size and/or location of a low-pressure area on a bed can be selected so that it is under a portion of a person's body which has a pressure ulcer or is at risk for getting a pressure ulcer. In this manner, that portion of the person's body will have less force exerted on it from the bed surface.

In an example, the size and/or location of a low-pressure area on a bed can be changed over time to avoid prolonged pressure on a particular portion of a person's body. In an example, the location of a low-pressure area can be moved like a wave across the surface of a bed. In an example, the location of a low-pressure area can be moved across the surface of a bed according to a programmed pattern. In an example, the location of a low-pressure area can be moved across the surface of a bed according to a programmed pattern which includes movement in a head-to-toe direction and movement in a right-to-left direction.

In this example, a segment of a strap has greater elasticity than the rest of the strap and elastic segments of overlapping straps are aligned to create a low-pressure area. In a variation on this example, a segment of a strap can be more stretchable than the rest of the strap and stretchable segments of overlapping straps can be aligned to create a low-pressure area. In a variation on this example, a segment of a strap can be thinner than the rest of the strap and thin segments of overlapping straps can be aligned to create a low-pressure area. In a variation on this example, a segment of a strap can have a lower durometer than the rest of the strap and low-durometer segments of overlapping straps can be aligned to create a low-pressure area. In a variation on this example, a segment of a strap can have an opening (or hole) and segments of overlapping straps with openings (or holes) can be aligned to create a low-pressure area.

In an example, this device can comprise at least one electromagnetic motor (and/or actuator) which moves one or more straps. In an example, at least one electromagnetic motor can be incorporated into a bed or mattress. In an example, at least one electromagnetic motor can be located inside the bed or mattress. In this example, there are a plurality of electromagnetic motors which move (e.g. shift) straps laterally across the surface of a bed or mattress. These motors are shown in FIG. 3, but are not shown in FIG. 4 to avoid diagrammatic clutter. In this example, there is a first set of motors which move (e.g. shift) straps in the first set of straps which spans a bed in a head-to-toe direction and a second set of motors which move (e.g. shift) straps in the second set of straps which spans a bed in a right-to-left direction. In FIG. 3, elastic (or thin) segments are all aligned along the headboard and the right side of the bed or mattress. In FIG. 4, elastic (or thin) segments of subsets of straps have been moved laterally (e.g. shifted) so as to overlap in area 401, creating a low-pressure area there.

In this example, there are two electromagnetic motors for each strap, one on each end of a strap. In an example, these electromagnetic motors can control the rotation of spools which wind, or unwind, each end of each strap. Alternatively, there can be one electromagnetic motor per strap. There can be a spool on only one end of a strap which winds, or unwinds, a strap. In another example, a strap can pass between two rollers which are moved by a motor, wherein rotation of the rollers moves the strap. In this example, a motor can move a strap laterally, thereby moving the locations of an elastic (or thin) strap segments. In an example, a strap can comprise a loop, wherein a portion of the loop spans the upper surface of a bed or mattress, wherein a portion of the loop spans the interior of the bed or mattress, and wherein a motor (acting on the interior portion) rotates the loop to change the location of an elastic (or thin) segment of the strap. Alternatively, an electromagnetic motor can control a piston which changes the length and/or tension of a strap. Alternatively, one electromagnetic motor can selectively move a plurality of straps via a set of mechanical gears or linkages.

In an example, the portions of straps which span the upper surface of a bed or mattress can loop around rollers on the sides of the bed or mattress and engage with one or more movement mechanisms (e.g. one or more motors and/or actuators) which are below the upper surface of the bed or mattress. In example, each strap can have its own roller on each side of the bed so that each strap can be moved independently. In an example, a roller can be a passive (non-mechanized) roller which reduces friction as a strap which loops around the side of a bed is moved by a motor below the surface of the bed or mattress. Alternative, a roller can be an active roller (e.g. directly moved by a motor) which actively engages and moves a strap. In an example, one or more ends of a strap can wind around one or more spools.

In an example, a strap can be a loop, wherein an upper portion of the loop spans the upper surface of a bed or mattress and a lower portion of the loop spans an interior portion of the bed or mattress. In an example, rollers can engage the loop and rotate it so that portions of the loop switch places, with portions which were on the surface now moving inside the bed or mattress and portions which were inside the bed or mattress now moving to the surface. In this manner, portions of the strap with greater elasticity can be moved to or from the surface, as well as to different locations on the surface, of the bed or mattress. In an example, this device can further comprise a drying and/or cleaning mechanism which dries and/or cleans a portion of a loop as that portion passes through the interior of a bed or mattress (e.g. a section of the bed or mattress other than its upper surface).

In an example, this device can further comprising a data processor which receives input data from human commands and/or sensors and selects which motors to activate to create a low-pressure area at a desired location based on this input data. In an example, this device can further comprise a wireless data transmitter by which the data processor is in wireless communication with a remote device such as a cell phone.

In an example, a device for reducing pressure ulcers by creating one or more low-pressure areas on a support surface on which a person rests can have three layers: an upper layer (such as a sheet) which is continuous, breathable, and flexible; a middle layer which is a woven orthogonal matrix of straps with elastic segments; and a lower level which is either air space or a highly-compressible foam. In an example, a device for reducing pressure ulcers by creating one or more low-pressure areas on a support surface on which a person rests can have four layers: an upper layer (such as a sheet) which is continuous, breathable, and flexible; a first middle layer comprising a first set of flexible parallel straps which spans a bed in a first direction; a second middle layer comprising a second set of flexible parallel straps which spans the bed in a second direction which is orthogonal to the first direction; and a lower level which is either air space or a highly-compressible foam.

This device with rows and columns of straps can enable adjustment of low-pressure areas of different sizes and/or locations across the surface of a bed with much less mechanical complexity than a device with a separate adjustment mechanism (such as an inflatable cell) for each area. For example, with 10 adjustment mechanisms to adjust 10 rows (e.g. 10 straps which span a bed in a right-to-left direction) and 10 mechanisms to adjust 10 columns (e.g. 10 straps which span the bed in a head-to-foot direction), one can selectively adjust the pressure of 100 different cells (overlap areas) on the bed. This only takes 20 adjustment mechanisms. In contrast, with one adjustment mechanism for each cell, it would take 100 adjustment mechanisms. This is why beds in the prior art with a matrix of individually-inflatable cells become mechanically complex very quickly. The smart bed or mattress disclosed herein offers less mechanical complexity. The bed of mattress disclosed herein also has a more air-permeable surface than a bed or mattress with inflatable cells. This is which is better for the person's skin health because it retains less moisture and heat than a bed or mattress with inflatable cells.

In an example, this device can be part of a system which automatically adjusts the size and/or location one or more low-pressure areas on a person's bed based on a predetermined pattern and/or schedule. In an example, this device can be part of a system which periodically varies the sizes and/or locations of one or more low-pressure areas on a person's bed in order to reduced prolonged periods of high pressure for any particular portion of the person's body.

In an example, this device can be part of a system which adjusts the size and/or location of one or more low-pressure areas on a person's bed based on commands from a human-to-computer interface. In an example, these commands can be voice commands from the resting person or from a caregiver. In an example, these commands can be touch-based (e.g. button or touch-screen) commands from the person or from a caregiver. In an example, such a system can adjust the size and/or location of one or more low-pressure areas in real time in response to feedback from the person (e.g. with respect to comfort). In an example, such a system can adjust the size and/or location of one or more low-pressure areas in response to progress in the healing of pressure ulcers.

In an example, this device can be part of a system which automatically adjusts the size and/or location of one or more low-pressure areas on a person's bed based on data from one or more sensors regarding the location of a portion of the person's body which is at risk for a pressure ulcer in order to prevent a pressure ulcer. In an example, this device can be part of a system which automatically adjusts the size and/or location of one or more low-pressure areas on a person's bed based on data from sensors regarding the location of a portion of the person's body with a pressure ulcer in order to help heal the pressure ulcer. In an example, these one or more sensors can be selected from the group consisting of: a camera which records images in the visible spectrum; an infrared camera; a plurality of electromagnetic (e.g. resistive or capacitive) stretch sensors; a plurality of pressure sensors on the upper surface of a bed or mattress; a garment with pressure sensors worn by the person; and a garment with electromagnetic energy emitters worn by the person.

In an example, this device can further comprise a camera whose images are analyzed to identify the location of a portion of a person's body which is particularly at risk for pressure ulcers so that a low-pressure area of the bed can be created under that portion. In an example, this device can further comprise an infrared imager whose images are analyzed to identify the location of a portion of a person's body which is particularly at risk for pressure ulcers so that a low-pressure area of the bed can be created under that portion. In an example, this device can further comprise an array of pressure sensors whose data is analyzed to identify the location of a portion of a person's body which is particularly at risk for pressure ulcers so that a low-pressure area of the bed can be created under that portion. Example variations discussed elsewhere in this disclosure or in priority-linked disclosures can also be applied to this example where relevant.

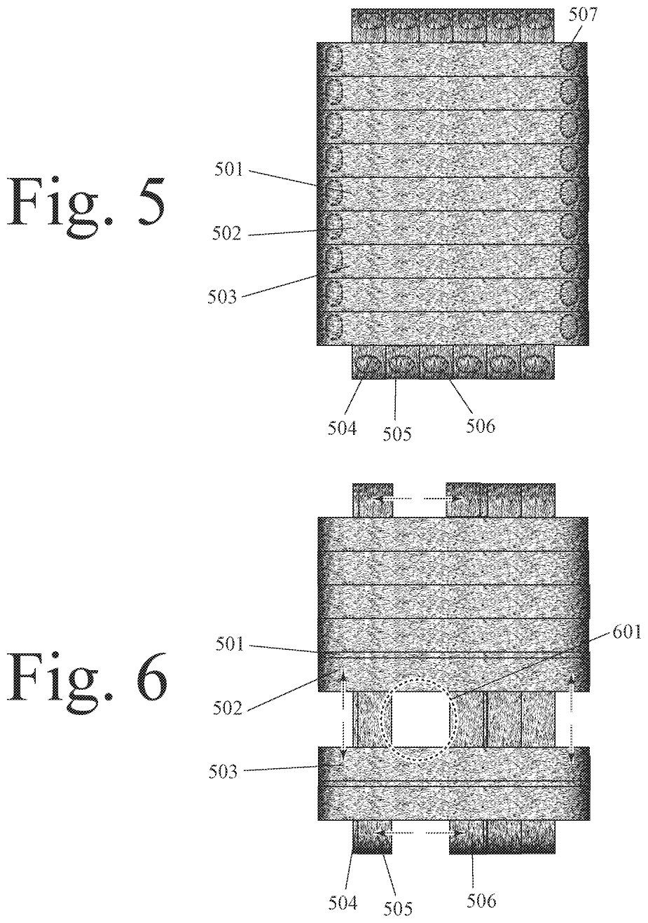

FIGS. 5 and 6 show two top-down views, at two different times, of another example of a smart bed or mattress for reducing pressure ulcers. Similar to previous examples in FIGS. 1 through 4, this example also includes an orthogonal array of straps. However, unlike in previous examples, straps in this present example slide sideways to leave an opening (or hole) in the upper surface of the smart bed or mattress. This opening (or hole) need not be completely empty. There can be a sheet over it. This sheet can protect the person's body from abrasion on the edges of the opening (or hole). Another difference in this example is that straps spanning the bed or mattress in different directions are overlaid in different layers, not woven together as in previous examples. Also, increased airflow through the opening (or hole) to a vulnerable portion of the person's body can also be an advantage of this design because it helps to reduce the accumulation of moisture or heat.

FIG. 5 shows this smart bed or mattress at a first point in time, before selected straps in an orthogonal array of straps have been selectively moved (e.g. slid sideways) to create a low-pressure area (e.g. an opening) on the bed or mattress. FIG. 6 shows this smart bed or mattress at a second point in time, after selected straps in the orthogonal array of straps have been selectively moved (e.g. slid sideways) to create a low-pressure area (e.g. an opening) on the bed or mattress. In this example, a low-pressure area (e.g. opening) in a support surface is created where straps spanning different directions which had overlapped in a given area are both moved (e.g. slid sideways) away from that area. The size and location of the low-pressure area (e.g. opening) is selected so as to reduce pressure on a portion of a person's body which is at high risk of getting a pressure ulcer or which already has a pressure ulcer.

The smart bed or mattress for reducing pressure ulcers which is shown in FIGS. 5 and 6 comprises: (a) a bed or mattress on which a person rests; (b) at least one electromagnetic motor; (c) a first set of flexible straps which span the bed or mattress in a head-to-foot direction; wherein a first subset of straps comprises one or more straps selected from the first set; wherein straps in the first subset are slid sideways by one or more motors selected from the at least one motor; wherein different first subsets are selected and slid at different times to create openings (or holes) with different sizes and/or locations in the upper surface of the bed or mattress; and wherein the selection of straps in the first subset is based at least partly on predetermined timing, a position of the person's body, analysis of camera images, data from infrared sensors, data from stretch sensors, and/or data from pressure sensors; and (d) a second set of flexible straps which span the bed or mattress in a right-to-left direction; wherein a second subset of straps comprises one or more straps selected from the second set; wherein straps in the second subset are slid sideways by one or more motors selected from the at least one motor; wherein different second subsets are selected and slid at different times to create openings (or holes) with different sizes and/or locations in the upper surface of the bed or mattress; and wherein the selection of straps in the second subset is based at least partly on predetermined timing, a position of the person's body, analysis of camera images, data from infrared sensors, data from stretch sensors, and/or data from pressure sensors; wherein a low-pressure area is an area on the bed or mattress where pressure exerted by the bed or mattress on the person's body is reduced relative to other areas of the bed or mattress; and wherein a low-pressure area is formed where the openings (or holes) created by movement of first and second subsets are aligned.

With respect to specific components, the smart bed or mattress shown in FIGS. 5 and 6 comprises: a plurality of electromagnetic motors (including 507); a first set of flexible straps (including 504, 505, and 506) which span the bed or mattress in a head-to-foot direction; wherein a first subset of straps (505 and 506) are moved (e.g. slid sideways) from a non-overlapping configuration (see FIG. 5) to an overlapping configuration (see FIG. 6); and a second set of flexible straps (including 501, 502, and 503) which span the bed or mattress in a right-to-left direction; wherein a second subset of straps (502 and 503) selected from the second set are moved (e.g. slid sideways) from a non-overlapping configuration (see FIG. 5) to an overlapping configuration (see FIG. 6); wherein a low-pressure area 601 is an area on the bed or mattress where pressure exerted by the bed or mattress on the person's body is reduced relative to other areas of the bed or mattress; and wherein the low-pressure area is formed where openings (or holes) created by movement of the first and second subsets are aligned. The motors are shown in FIG. 5, but not also shown in FIG. 6 in order to reduce diagrammatic clutter.

In an example, straps can span the upper surface of a bed or mattress. In an example, straps can be close to the upper surface of a bed or mattress, but there can be a sheet (or other flexible layer) between the straps and a person's body. In an example, a sheet (or other flexible layer) between the straps and a person's body can protect the person from abrasion when the straps move and can protect the person from being caught on the edges of an opening (or hole). In an example, straps or a sheet (or other flexible layer) can further comprise an array of pressure sensors whose data is analyzed to measure area-specific pressure levels of a person's body on a strap matrix. In an example, the device can further comprise a lift mechanism which temporarily lifts a person up before the straps move and lowers the person down after the straps have moved.

In an example, this device can comprise a smart bed or smart mattress. In an example, this device can comprise a smart mattress pad, smart sheet, or smart blanket. In an example, this device can comprise a flexible net, mesh, sling, or hammock. In an example, this device can be a stand-alone product. In an example, this device can be used in combination with an existing bed or mattress. In an example, this device can be used on top of an existing bed or mattress. In an example, straps can be made from a strong, but also flexible and breathable, fabric. In variations on this example, strips, bands, belts, loops, or cords can be used instead of straps.

In an example, straps in a first set of straps can be parallel to each other. In an example, straps in a second set of straps can be parallel to each other. In an example, straps in a second set of straps can be orthogonal to straps in a first set of straps. In an example, straps in a second set of straps can overlay straps in a first set of straps. In an example, a low-pressure area can be formed from alignment of multiple openings (or holes). In an example, a low-pressure area can have a first side comprising multiple strap widths and a second side comprising multiple strap widths. In an example, the size and/or location of a low-pressure area on a bed or mattress can be selected so that it is under a portion of a person's body which has a pressure ulcer or is at risk for getting a pressure ulcer. In this manner, that portion of the person's body will have less force exerted on it from the bed or mattress surface.

In an example, straps can be between 2 inches and 2 feet wide. In an example, a first set of straps which spans a bed in a head-to-foot direction can comprise at least 4 straps. In an example, a first set of straps which spans a bed in a head-to-foot direction can comprise between 3 and 10 straps. In an example, a second set of straps which spans a bed in a right-to-left direction can comprise at least 5 straps. In an example, a second set of straps which spans a bed in a right-to-left direction can comprise between 4 and 15 straps. In an example, a first set of straps which spans a bed in a head-to-foot direction can comprise at least 4 straps, a second set of straps which spans a bed in a right-to-left direction can comprise at least 5 straps, and there can be at least (4×5=) 20 areas of overlap between the first and second sets wherein low-pressure areas can be selectively formed. In an example, a first set of straps which spans a bed in a head-to-foot direction can comprise at least 10 straps, a second set of straps which spans a bed in a right-to-left direction can comprise at least 10 straps, and there can be at least (10×10=) 100 areas of overlap between the first and second sets wherein low-pressure areas can be selectively formed.

In an example, the size and/or location of a low-pressure area on a bed can be changed over time to avoid prolonged pressure on a particular portion of a person's body. In an example, the location of a low-pressure area can be moved like a wave across the surface of a bed. In an example, the location of a low-pressure area can be moved across the surface of a bed according to a programmed pattern. In an example, the location of a low-pressure area can be moved across the surface of a bed according to a programmed pattern which includes movement in a head-to-toe direction and movement in a right-to-left direction.

In an example, this device can comprise at least one electromagnetic motor (and/or actuator) which moves one or more straps. In an example, at least one electromagnetic motor can be incorporated into a bed or mattress. In an example, at least one electromagnetic motor can be located inside a bed or mattress. In this example, there are a plurality of electromagnetic motors which move straps. In this example, there is a first set of motors which move (e.g. slide sideways) straps in the first set of straps which spans a bed in a head-to-toe direction and a second set of motors which move (e.g. slide sideways) straps in the second set of straps which spans a bed in a right-to-left direction.

In an example, flexible straps can be individually and independently moved. In an example, flexible straps in first and second sets can be analogous to a matrix with columns and rows, wherein a particular cell in the matrix can be activated by activating a selected column and row. In an example, this device can further comprising a data processor which receives input data from human commands and/or sensors and selects which motors to activate to create a low-pressure area at a desired location based on this input data. In an example, this device can further comprise a wireless data transmitter by which the data processor is in wireless communication with a remote device such as a cell phone.

In an example, a device for reducing pressure ulcers by creating one or more low-pressure areas on a support surface on which a person rests can have four layers: an upper layer (such as a sheet) which is continuous, breathable, and flexible; a first middle layer comprising a first set of flexible parallel straps which spans a bed in a first direction; a second middle layer comprising a second set of flexible parallel straps which spans the bed in a second direction which is orthogonal to the first direction; and a lower level which is air space or a highly-compressible foam.

This device with rows and columns of straps can enable adjustment of low-pressure areas of different sizes and/or locations across the surface of a bed with much less mechanical complexity than a device with a separate adjustment mechanism (such as an inflatable cell or individual piston cell) for each area. For example, with 10 adjustment mechanisms to adjust 10 rows (e.g. 10 straps which span a bed in a right-to-left direction) and 10 mechanisms to adjust 10 columns (e.g. 10 straps which span the bed in a head-to-foot direction), one can selectively and individually adjust the pressure of 100 different cells (overlap areas) on the bed. This only takes 20 adjustment mechanisms. In contrast, with a device with one adjustment mechanism for each cell, it would take 100 adjustment mechanisms. This is why most beds in the prior art with a matrix of individually-inflatable cells become mechanically complex very quickly. The device disclosed herein offers less mechanical complexity than devices with individual inflatable cells. The bed disclosed herein also has a more air-permeable surface which is better for skin health than a bed with inflatable cells which can retain moisture and heat.

In an example, this device can be part of a system which automatically adjusts the size and/or location one or more low-pressure areas on a person's bed based on a predetermined pattern and/or schedule. In an example, this device can be part of a system which periodically varies the sizes and/or locations of one or more low-pressure areas on a person's bed in order to reduced prolonged periods of high pressure for any particular portion of the person's body. In an example, adjacent areas of a bed or mattress can alternate over time between a normal-pressure configuration and a low-pressure configuration.

In an example, this device can be part of a system which adjusts the size and/or location of one or more low-pressure areas on a person's bed or mattress based on commands from a human-to-computer interface. In an example, these commands can be voice commands from the person or a caregiver. In an example, these commands can be touch-based (e.g. button or touch-screen) commands from the resting person or from a caregiver. In an example, such a system can adjust the size and/or location of one or more low-pressure areas in real time in response to feedback from the resting person (e.g. with respect to comfort). In an example, such a system can adjust the size and/or location of one or more low-pressure areas in response to documented progress in the healing of pressure ulcers (e.g. reinforcing pressure patterns associated with healing and reducing patterns associated with pathology).

In an example, this device can be part of a system which automatically adjusts the size and/or location one or more low-pressure areas on a person's bed or mattress based on data from one or more sensors. In an example, these sensors can indicated the location of a portion of the person's body which is at risk for a pressure ulcer and a low-pressure area can be created underneath than portion in order to prevent a pressure ulcer. In an example, this device can be part of a system which automatically adjusts the size and/or location a low-pressure area on a person's bed based on data from sensors regarding the location of a portion of the person's body with a pressure ulcer in order to help heal the pressure ulcer. In an example, these one or more sensors can be selected from the group consisting of: a camera which records images in the visible spectrum; an infrared camera and/or sensor; a plurality of electromagnetic (e.g. resistive or capacitive) stretch or bend sensors which are integrated into the bed, mattress, or a sheet; a plurality of pressure sensors on the upper surface of a bed or mattress; a plurality of pressure sensors on a garment worn by the person; and a garment with electromagnetic energy emitters worn by the person whose signals are used to identify the person's body configuration.

In an example, this device can further comprise a video camera whose images are analyzed to recognize a person's body configuration on a bed or mattress and thereby identify the locations of one or more portions of the person's body which at risk for pressure ulcers. In an example, one or more low-pressure areas can be created under those one or more portions. In an example, this device can further comprise an infrared imager whose data is analyzed to recognize a person's body configuration on a bed or mattress and thereby identify the locations of one or more portions of the person's body which at risk for pressure ulcers. In an example, one or more low-pressure areas can be created under those one or more portions.

In an example, this device can further comprise a plurality (e.g. an array, grid, or matrix) of pressure sensors whose data is analyzed to recognize a person's body configuration on a bed or mattress and thereby identify the locations of one or more portions of the person's body which at risk for pressure ulcers. In an example, one or more low-pressure areas can be created under those one or more portions. In an example, this device can further comprise a plurality (e.g. an array, grid, or matrix) of stretch and/or bend sensors whose data is analyzed to recognize a person's body configuration on a bed or mattress and thereby identify the locations of one or more portions of the person's body which at risk for pressure ulcers. In an example, one or more low-pressure areas can be created under those one or more portions. Example variations discussed elsewhere in this disclosure or in priority-linked disclosures can also be applied to this example where relevant.

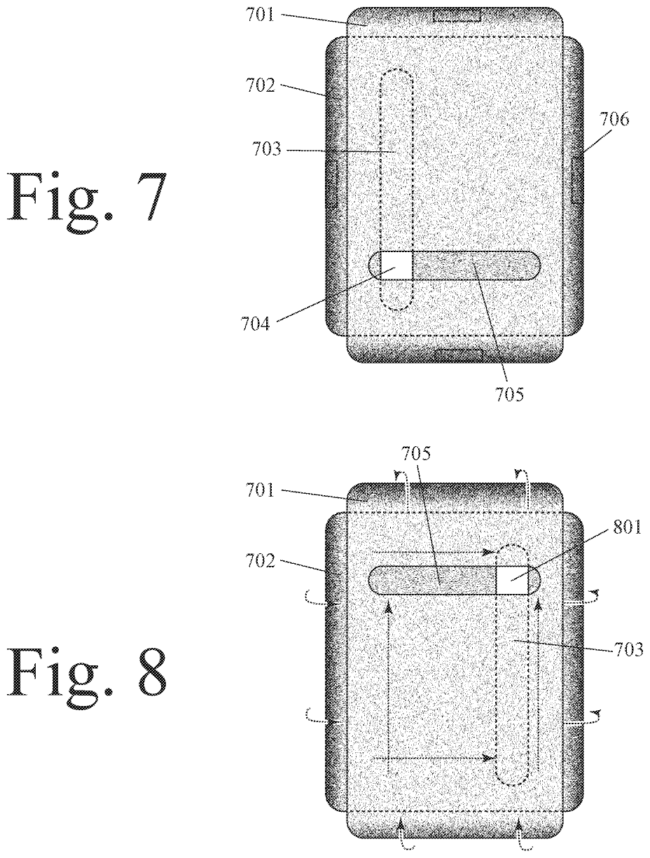

FIGS. 7 and 8 show two top-down views, at two different times, of another example of a smart bed or mattress for reducing pressure ulcers. In this example, a body support surface has two moveable layers, an upper layer which spans the bed or mattress in a first direction and a lower layer which spans the bed or mattress in a second direction which is orthogonal to the first direction. Each of the two layers has an opening (or hole) in it. The area where the openings (or holes) in the two layers are aligned is a low-pressure area. The location of the low-pressure area can be selectively moved to different places on the body support surface by selectively moving the two layers relative to each other.

FIG. 7 shows this smart bed or mattress at a first point in time when openings (or holes) in two layers are aligned at a first location. FIG. 8 shows this smart bed or mattress at a second point in time when the two layers have each been moved and these openings (or holes) are now aligned at a second location. A low-pressure area (e.g. opening) in a body support surface is created where the openings (or holes) align. The size and location of the low-pressure area (e.g. opening) is selected so as to reduce pressure on a portion of a person's body which is at high risk of getting a pressure ulcer or which already has a pressure ulcer.

The smart bed or mattress for reducing pressure ulcers which is shown in FIGS. 7 and 8 comprises: (a) a bed or mattress on which a person rests; (b) at least one electromagnetic motor; (c) a first support layer which spans the bed or mattress in a head-to-foot direction, wherein there is a first opening (or hole) in the first support layer; and (d) a second support layer which spans the bed or mattress in a right-to-left direction, wherein there is a second opening (or hole) in the second support layer, wherein the first support layer and the second support layer are selectively moved by the at least one electromagnetic motor so as to align the first opening and the second opening at a selected location on the bed or mattress, thereby creating a low-pressure area at the selected location, and wherein the selected location is selected at least partly based on predetermined timing, a position of the person's body, analysis of camera images, data from infrared sensors, data from stretch sensors, and/or data from pressure sensors.

With respect to specific components, the smart bed or mattress shown in FIGS. 7 and 8 comprises: at least one electromagnetic motor 706; a first support layer 701 which spans the bed or mattress in a head-to-foot direction, wherein there is a first opening (or hole) 705 in the first support layer; and a second support layer 702 which spans the bed or mattress in a right-to-left direction, wherein there is a second opening (or hole) 703 in the second support layer, wherein the first support layer and the second support layer are selectively moved by the at least one electromagnetic motor so as to align the first opening and the second opening at a selected location on the bed or mattress, thereby creating a low-pressure area (e.g. opening or hole) 704 at the selected location, and wherein the selected location is selected at least partly based on predetermined timing, a position of the person's body, analysis of camera images, data from infrared sensors, data from stretch sensors, and/or data from pressure sensors. In this example, the first support layer is above the second support layer. Alternatively, the second support layer could be above the first support layer. The motor is not shown in FIG. 8 in order to reduce diagrammatic clutter.

In an example, there can be a sheet (or other flexible layer) between the movable layers and a person's body. In an example, a sheet (or other flexible layer) between the layers and a person's body can protect the person from abrasion when the layers move and can protect the person from being caught on the edges of an opening (or hole). In an example, layers or a sheet (or other flexible layer) can further comprise an array of pressure sensors whose data is analyzed to measure area-specific pressure levels of a person's body on a layer matrix. In an example, the device can further comprise a lift mechanism which temporarily lifts a person up before the layers move and lowers the person down after the layers have moved.

In an example, this device can comprise a smart bed or smart mattress. In an example, this device can comprise a smart mattress pad, smart sheet, or smart blanket. In an example, this device can comprise a flexible net, mesh, sling, or hammock. In an example, this device can be a stand-alone product. In an example, this device can be used in combination with an existing bed or mattress. In an example, this device can be used on top of an existing bed or mattress. In an example, layers can be made from a strong, but also flexible and breathable, fabric.

In an example, the size and/or location of a low-pressure area (e.g. opening or hole) on a bed can be changed over time to avoid prolonged pressure on a particular portion of a person's body. In an example, the location of a low-pressure area (e.g. opening or hole) can be moved like a wave across the surface of a bed. In an example, the location of a low-pressure area (e.g. opening or hole) can be moved across the surface of a bed according to a programmed pattern. In an example, the location of a low-pressure area (e.g. opening or hole) can be moved across the surface of a bed according to a programmed pattern which includes movement in a head-to-toe direction and movement in a right-to-left direction.

In an example, this device can further comprising a data processor which receives input data from human commands and/or sensors and selects how to move the layers to activate to create a low-pressure area at a desired location based on this input data. In an example, this device can further comprise a wireless data transmitter by which the data processor is in wireless communication with a remote device such as a cell phone.

In an example, a device for reducing pressure ulcers by creating one or more low-pressure areas on a support surface on which a person rests can have four layers: a sheet which is continuous, breathable, and flexible; an first moving layer (with an opening) which spans a bed in a first direction; a second moving layer (with an opening) which spans the bed in a second direction which is orthogonal to the first direction; and air space or highly-compressible foam.

In an example, this device can be part of a system which automatically adjusts the size and/or location one or more low-pressure areas on a person's bed based on a predetermined pattern and/or schedule. In an example, this device can be part of a system which periodically varies the sizes and/or locations of one or more low-pressure areas on a person's bed in order to reduced prolonged periods of high pressure for any particular portion of the person's body. In an example, adjacent areas of a bed or mattress can alternate over time between a normal-pressure configuration and a low-pressure configuration.

In an example, this device can be part of a system which adjusts the size and/or location of one or more low-pressure areas on a person's bed or mattress based on commands from a human-to-computer interface. In an example, these commands can be voice commands from the person or a caregiver. In an example, these commands can be touch-based (e.g. button or touch-screen) commands from the resting person or from a caregiver. In an example, such a system can adjust the size and/or location of one or more low-pressure areas in real time in response to feedback from the resting person (e.g. with respect to comfort). In an example, such a system can adjust the size and/or location of one or more low-pressure areas in response to documented progress in the healing of pressure ulcers (e.g. reinforcing pressure patterns associated with healing and reducing patterns associated with pathology).

In an example, this device can be part of a system which automatically adjusts the size and/or location one or more low-pressure areas on a person's bed or mattress based on data from one or more sensors. In an example, these sensors can indicated the location of a portion of the person's body which is at risk for a pressure ulcer and a low-pressure area can be created underneath than portion in order to prevent a pressure ulcer. In an example, this device can be part of a system which automatically adjusts the size and/or location a low-pressure area on a person's bed based on data from sensors regarding the location of a portion of the person's body with a pressure ulcer in order to help heal the pressure ulcer. In an example, these one or more sensors can be selected from the group consisting of: a camera which records images in the visible spectrum; an infrared camera and/or sensor; a plurality of electromagnetic (e.g. resistive or capacitive) stretch or bend sensors which are integrated into the bed, mattress, or a sheet; a plurality of pressure sensors on the upper surface of a bed or mattress; a plurality of pressure sensors on a garment worn by the person; and a garment with electromagnetic energy emitters worn by the person whose signals are used to identify the person's body configuration.

In an example, this device can further comprise a video camera whose images are analyzed to recognize a person's body configuration on a bed or mattress and thereby identify the locations of one or more portions of the person's body which at risk for pressure ulcers. In an example, one or more low-pressure areas can be created under those one or more portions. In an example, this device can further comprise an infrared imager whose data is analyzed to recognize a person's body configuration on a bed or mattress and thereby identify the locations of one or more portions of the person's body which at risk for pressure ulcers. In an example, one or more low-pressure areas can be created under those one or more portions.

In an example, this device can further comprise a plurality (e.g. an array, grid, or matrix) of pressure sensors whose data is analyzed to recognize a person's body configuration on a bed or mattress and thereby identify the locations of one or more portions of the person's body which at risk for pressure ulcers. In an example, one or more low-pressure areas can be created under those one or more portions. In an example, this device can further comprise a plurality (e.g. an array, grid, or matrix) of stretch and/or bend sensors whose data is analyzed to recognize a person's body configuration on a bed or mattress and thereby identify the locations of one or more portions of the person's body which at risk for pressure ulcers. In an example, one or more low-pressure areas can be created under those one or more portions. Example variations discussed elsewhere in this disclosure or in priority-linked disclosures can also be applied to this example where relevant.

Other example variations are now discussed. In an example, a device to prevent pressure ulcers can comprise: a first plurality of parallel flexible strips; a second plurality of parallel flexible strips; wherein the first and second pluralities of parallel flexible strips are woven together to create an orthogonal mesh or grid which supports a sleeping person's body; and a plurality of actuators which move selected strips apart from each other in order to create a quadrilateral-shaped opening in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a first plurality of parallel flexible strips; a second plurality of parallel flexible strips; wherein the first and second pluralities of parallel flexible strips are woven together to create an orthogonal mesh or grid which supports a sleeping person's body; and a plurality of actuators which move selected strips apart from each other in order to enlarge a quadrilateral-shaped opening in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a first plurality of parallel flexible strips; a second plurality of parallel flexible strips; a third plurality of parallel flexible strips; wherein the first, second, and third pluralities of parallel flexible strips are woven together to create a hexagonal mesh or grid which supports a sleeping person's body; and a plurality of actuators which move selected strips apart from each other in order to enlarge a hexagonal-shaped opening in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a first plurality of parallel flexible strips; a second plurality of parallel flexible strips; a third plurality of parallel flexible strips; wherein the first, second, and third pluralities of parallel flexible strips are woven together to create a hexagonal mesh or grid which supports a sleeping person's body; and a plurality of actuators which move selected strips apart from each other in order to create a hexagonal-shaped opening in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a first roller (or tube) at the head a bed (or bed mattress); a second roller (or tube) at the foot of the bed; one or more actuators; and a plurality of moving loops, wherein an upper portion of each loop spans the length of the bed between the rollers in a first direction; wherein a lower portion of each moving loop spans the length of the bed between the rollers in a second direction; and wherein each loop is rotated around the rollers by the one or more actuators.

In an example, a device to prevent pressure ulcers can comprise: a first roller (or tube) at the head a bed (or bed mattress); a second roller (or tube) at the foot of the bed; one or more actuators; and a plurality of moving loops, wherein each loop has longitudinal variation in thickness, wherein an upper portion of each loop spans the length of the bed between the rollers in a first direction; wherein a lower portion of each moving loop spans the length of the bed between the rollers in a second direction;

In an example, a device to prevent pressure ulcers can comprise: a first roller (or tube) at the head a bed (or bed mattress); a second roller (or tube) at the foot of the bed; one or more actuators; and a plurality of moving loops, wherein an upper portion of each loop spans the length of the bed between the rollers in a first direction; wherein a lower portion of each moving loop spans the length of the bed between the rollers in a second direction; wherein each loop is rotated around the rollers by the one or more actuators; and wherein adjacent loops are moved and/or rotated in opposite longitudinal directions.

In an example, a device to prevent pressure ulcers can comprise: a first roller (or tube) at the head a bed (or bed mattress); a second roller (or tube) at the foot of the bed; one or more actuators; and a plurality of moving undulating loops, wherein each loop has longitudinal undulation in thickness, wherein an upper portion of each loop spans the length of the bed between the rollers in a first direction; wherein a lower portion of each moving loop spans the length of the bed between the rollers in a second direction; wherein each loop is rotated around the rollers by the one or more actuators; and wherein adjacent loops are moved and/or rotated in opposite longitudinal directions.

In an example, a device to prevent pressure ulcers can comprise: a first roller (or tube) at the head a bed (or bed mattress); a second roller (or tube) at the foot of the bed; one or more actuators; and a plurality of moving sinusoidal loops, wherein each loop has sinusoidal variation in thickness, wherein an upper portion of each loop spans the length of the bed between the rollers in a first direction; wherein a lower portion of each moving loop spans the length of the bed between the rollers in a second direction; wherein each loop is rotated around the rollers by the one or more actuators; and wherein adjacent loops are moved and/or rotated in opposite longitudinal directions.

In an example, a device to prevent pressure ulcers can comprise: a first roller (or tube) at the head a bed (or bed mattress); a second roller (or tube) at the foot of the bed; one or more actuators; and a plurality of moving sinusoidal loops, wherein each loop has sinusoidal variation in thickness, wherein an upper portion of each loop spans the length of the bed between the rollers in a first direction; wherein a lower portion of each moving loop spans the length of the bed between the rollers in a second direction; and wherein each loop is rotated around the rollers by the one or more actuators.

In an example, a device to prevent pressure ulcers can comprise: a first roller (or tube) at the head a bed (or bed mattress); a second roller (or tube) at the foot of the bed; one or more actuators; and a plurality of moving undulating loops, wherein each loop has longitudinal undulation in thickness, wherein an upper portion of each loop spans the length of the bed between the rollers in a first direction; wherein a lower portion of each moving loop spans the length of the bed between the rollers in a second direction; and wherein each loop is rotated around the rollers by the one or more actuators.

In an example, a device to prevent pressure ulcers can comprise: a first roller (or tube) at the head a bed (or bed mattress); a second roller (or tube) at the foot of the bed; one or more actuators; and a plurality of moving sinusoidal loops, wherein each loop has sinusoidal variation in thickness, wherein an upper portion of each loop spans the length of the bed between the rollers in a first direction; wherein a lower portion of each moving loop spans the length of the bed between the rollers in a second direction; wherein each loop is rotated around the rollers by the one or more actuators; and wherein different loops are moved and/or rotated in different directions.

In an example, a device to prevent pressure ulcers can comprise: a first roller (or tube) at the head a bed (or bed mattress); a second roller (or tube) at the foot of the bed; one or more actuators; and a plurality of moving undulating loops, wherein each loop has longitudinal undulation in thickness, wherein an upper portion of each loop spans the length of the bed between the rollers in a first direction; wherein a lower portion of each moving loop spans the length of the bed between the rollers in a second direction; wherein each loop is rotated around the rollers by the one or more actuators; and wherein different loops are moved and/or rotated in different directions.

In an example, a device to prevent pressure ulcers can comprise: a first roller (or tube) at the head a bed (or bed mattress); a second roller (or tube) at the foot of the bed; one or more actuators; and a plurality of moving loops, wherein each loop has longitudinal variation in thickness, wherein an upper portion of each loop spans the length of the bed between the rollers in a first direction; wherein a lower portion of each moving loop spans the length of the bed between the rollers in a second direction; and wherein each loop is rotated around the rollers by the one or more actuators.

In an example, a device to prevent pressure ulcers can comprise: a first roller (or tube) at the head a bed (or bed mattress); a second roller (or tube) at the foot of the bed; one or more actuators; and a plurality of moving loops, wherein each loop has longitudinal variation in thickness, wherein an upper portion of each loop spans the length of the bed between the rollers in a first direction; wherein a lower portion of each moving loop spans the length of the bed between the rollers in a second direction; wherein each loop is rotated around the rollers by the one or more actuators; and wherein adjacent loops are moved and/or rotated in opposite longitudinal directions.

In an example, a device to prevent pressure ulcers can comprise: a first roller (or tube) at the head a bed (or bed mattress); a second roller (or tube) at the foot of the bed; one or more actuators; and a plurality of moving loops, wherein an upper portion of each loop spans the length of the bed between the rollers in a first direction; wherein a lower portion of each moving loop spans the length of the bed between the rollers in a second direction; wherein each loop is rotated around the rollers by the one or more actuators; and wherein different loops are moved and/or rotated in different directions.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving loops, wherein each loop has lateral variation in thickness, wherein an upper portion of each loop spans the width of the bed between the rollers in a first direction; wherein a lower portion of each moving loop spans the width of the bed between the rollers in a second direction; and wherein each loop is rotated around the rollers by the one or more actuators.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving sinusoidal loops, wherein each loop has sinusoidal variation in thickness, wherein an upper portion of each loop spans the width of the bed between the rollers in a first direction; wherein a lower portion of each moving loop spans the width of the bed between the rollers in a second direction; and wherein each loop is rotated around the rollers by the one or more actuators.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving undulating loops, wherein each loop has lateral undulation in thickness, wherein an upper portion of each loop spans the width of the bed between the rollers in a first direction; wherein a lower portion of each moving loop spans the width of the bed between the rollers in a second direction; and wherein each loop is rotated around the rollers by the one or more actuators.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving loops, wherein an upper portion of each loop spans the width of the bed between the rollers in a first direction; wherein a lower portion of each moving loop spans the width of the bed between the rollers in a second direction; wherein each loop is rotated around the rollers by the one or more actuators; and wherein different loops are moved and/or rotated in different directions.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving sinusoidal loops, wherein each loop has sinusoidal variation in thickness, wherein an upper portion of each loop spans the width of the bed between the rollers in a first direction; wherein a lower portion of each moving loop spans the width of the bed between the rollers in a second direction; wherein each loop is rotated around the rollers by the one or more actuators; and wherein different loops are moved and/or rotated in different directions.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving undulating loops, wherein each loop has lateral undulation in thickness, wherein an upper portion of each loop spans the width of the bed between the rollers in a first direction; wherein a lower portion of each moving loop spans the width of the bed between the rollers in a second direction; wherein each loop is rotated around the rollers by the one or more actuators; and wherein different loops are moved and/or rotated in different directions.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving sinusoidal loops, wherein each loop has sinusoidal variation in thickness, wherein an upper portion of each loop spans the width of the bed between the rollers in a first direction; wherein a lower portion of each moving loop spans the width of the bed between the rollers in a second direction; wherein each loop is rotated around the rollers by the one or more actuators; and wherein adjacent loops are moved and/or rotated in opposite lateral directions.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving loops, wherein each loop has lateral variation in thickness, wherein an upper portion of each loop spans the width of the bed between the rollers in a first direction; wherein a lower portion of each moving loop spans the width of the bed between the rollers in a second direction; wherein each loop is rotated around the rollers by the one or more actuators; and wherein different loops are moved and/or rotated in different directions.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving loops, wherein each loop has lateral variation in thickness, wherein an upper portion of each loop spans the width of the bed between the rollers in a first direction; wherein a lower portion of each moving loop spans the width of the bed between the rollers in a second direction; wherein each loop is rotated around the rollers by the one or more actuators; and wherein adjacent loops are moved and/or rotated in opposite lateral directions.

In an example, a device to prevent pressure ulcers can comprise: a first roller (or tube) on a first (e.g. right) side of a bed (or bed mattress); a second roller (or tube) on a second (e.g. left) side of the bed; one or more actuators; and a plurality of moving loops, wherein an upper portion of each loop spans the width of the bed between the rollers in a first direction; wherein a lower portion of each moving loop spans the width of the bed between the rollers in a second direction; and wherein each loop is rotated around the rollers by the one or more actuators.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first stretchability level and an arcuate (e.g. circular) second surface portion with a second stretchability level, wherein the second stretchability level is greater than the first stretchability level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third stretchability level and an arcuate (e.g. circular) fourth surface portion with a fourth stretchability level, wherein the fourth stretchability level is greater than the third stretchability level, wherein the fourth surface portion is smaller than (and nested within) the third surface portion, and wherein middle support surface and the lower support surface are moved so as to overlap the second surface portion and the fourth surface portion, thereby creating a localized surface portion with greater stretchability below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first durometer level and an arcuate (e.g. circular) second surface portion with a second durometer level, wherein the second durometer level is lower than the first durometer level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third durometer level and an arcuate (e.g. circular) fourth surface portion with a fourth durometer level, wherein the fourth durometer level is lower than the third durometer level, wherein the fourth surface portion is smaller than (and nested within) the third surface portion, and wherein middle support surface and the lower support surface are moved so as to overlap the second surface portion and the fourth surface portion, thereby creating a localized surface portion with lower durometer below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first durometer level and a second surface portion with a second durometer level, wherein the second durometer level is lower than the first durometer level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third durometer level and a fourth surface portion with a fourth durometer level, wherein the fourth durometer level is lower than the third durometer level, wherein the fourth surface portion is smaller than (and nested within) the third surface portion, and wherein middle support surface and the lower support surface are moved so as to overlap the second surface portion and the fourth surface portion, thereby creating a localized surface portion with lower durometer below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first flexibility level and an arcuate (e.g. circular) second surface portion with a second flexibility level, wherein the second flexibility level is greater than the first flexibility level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third flexibility level and an arcuate (e.g. circular) fourth surface portion with a fourth flexibility level, wherein the fourth flexibility level is greater than the third flexibility level, wherein the fourth surface portion is smaller than (and nested within) the third surface portion, and wherein middle support surface and the lower support surface are moved so as to overlap the second surface portion and the fourth surface portion, thereby creating a localized surface portion with greater stretchability below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first stretchability level and a second surface portion with a second stretchability level, wherein the second stretchability level is greater than the first stretchability level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third stretchability level and a fourth surface portion with a fourth stretchability level, wherein the fourth stretchability level is greater than the third stretchability level, wherein the fourth surface portion is smaller than (and nested within) the third surface portion, and wherein middle support surface and the lower support surface are moved so as to overlap the second surface portion and the fourth surface portion, thereby creating a localized surface portion with greater stretchability below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first elasticity level and an arcuate (e.g. circular) second surface portion with a second elasticity level, wherein the second elasticity level is greater than the first elasticity level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third elasticity level and an arcuate (e.g. circular) fourth surface portion with a fourth elasticity level, wherein the fourth elasticity level is greater than the third elasticity level, wherein the fourth surface portion is smaller than (and nested within) the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first flexibility level and a second surface portion with a second flexibility level, wherein the second flexibility level is greater than the first flexibility level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third flexibility level and a fourth surface portion with a fourth flexibility level, wherein the fourth flexibility level is greater than the third flexibility level, wherein the fourth surface portion is smaller than (and nested within) the third surface portion, and wherein middle support surface and the lower support surface are moved so as to overlap the second surface portion and the fourth surface portion, thereby creating a localized surface portion with greater stretchability below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is suspended between first and second rollers; wherein the middle support surface is manually moved between the first and second rollers, wherein the middle support surface has a first surface portion with a first elasticity level and a second surface portion with a second elasticity level, wherein the second elasticity level is greater than the first elasticity level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is suspended between third and fourth rollers; wherein the lower support surface is manually moved between the third and fourth rollers, wherein the middle support surface has a third surface portion with a third elasticity level and a fourth surface portion with a fourth elasticity level, wherein the fourth elasticity level is greater than the third elasticity level, wherein the fourth surface portion is smaller than (and nested within) the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is suspended between first and second rollers; wherein the middle support surface is automatically moved between the first and second rollers by one or more actuators, wherein the middle support surface has a first surface portion with a first elasticity level and a second surface portion with a second elasticity level, wherein the second elasticity level is greater than the first elasticity level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is suspended between third and fourth rollers; wherein the lower support surface is automatically moved between the third and fourth rollers, wherein the middle support surface has a third surface portion with a third elasticity level and a fourth surface portion with a fourth elasticity level, wherein the fourth elasticity level is greater than the third elasticity level, wherein the fourth surface portion is smaller than (and nested within) the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is suspended between first and second rollers; wherein the middle support surface is automatically moved between the first and second rollers by one or more actuators, wherein the middle support surface has a first opening (or hole); and a flexible and breathable (e.g. porous to gas and liquid) lower support surface (e.g. sheet, pad, blanket, net, or mesh) below the middle support surface, wherein the lower support surface is suspended between third and fourth rollers; wherein the lower support surface is automatically moved between the third and fourth rollers, wherein the middle support surface has a second opening (or hole), and wherein middle support surface and the lower support surface are moved so as to overlap the first and second openings (or holes), thereby creating a localized opening (or hole) below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first opening (or hole); and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a second opening (or hole), and wherein middle support surface and the lower support surface are moved so as to overlap the first opening (or hole) and the second opening (or hole), thereby creating a localized opening (or hole) below a selected part of the sleeping person's body that has a pressure ulcer and/or is particularly vulnerable to developing a pressure ulcer.

In an example, a device to prevent pressure ulcers can comprise: a flexible and breathable (e.g. porous to gas and liquid) upper support surface (e.g. sheet, pad, blanket, net, or mesh) on which a sleeping person lies; a flexible and breathable (e.g. porous to gas and liquid) middle support surface (e.g. sheet, pad, blanket, net, or mesh) below the upper support surface, wherein the middle support surface is automatically moved along a first (e.g. longitudinal or lateral) axis by one or more actuators, wherein the middle support surface has a first surface portion with a first elasticity level and a second surface portion with a second elasticity level, wherein the second elasticity level is greater than the first elasticity level, and wherein the second surface portion is smaller than (and nested within) the first surface portion; and a flexible and breathable (e.g. porous to gas and liquid) lower support surface (e.g. sheet, pad, blanket, net, or mesh) below the middle support surface, wherein the lower support surface is automatically moved along a second axis by one or more actuators, wherein the second axis is perpendicular to the first axis, wherein the middle support surface has a third surface portion with a third elasticity level and a fourth surface portion with a fourth elasticity level, wherein the fourth elasticity level is greater than the third elasticity level, wherein the fourth surface portion is smaller than (and nested within) the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: a flexible and breathable upper support surface on which a sleeping person lies; a flexible and breathable middle support surface below the upper support surface, wherein the middle support surface is automatically moved along a first axis by one or more actuators, wherein the middle support surface has a first surface portion with a first elasticity level and a second surface portion with a second elasticity level, wherein the second elasticity level is greater than the first elasticity level, and wherein the second surface portion is smaller than the first surface portion; and a flexible and breathable lower support surface below the middle support surface, wherein the lower support surface is automatically moved along a second axis by one or more actuators, wherein the second axis is perpendicular to the first axis, wherein the middle support surface has a third surface portion with a third elasticity level and a fourth surface portion with a fourth elasticity level, wherein the fourth elasticity level is greater than the third elasticity level, wherein the fourth surface portion is smaller than the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: a flexible support surface (such as a flexible net, mesh, sling, hammock, pad, sheet, or blanket) on which a person lies; a longitudinally-scrolling loop of padding (such as a loop of compressible foam or a loop of compressible textile padding) which is under the upper support structure; two rollers, one roller closer to the head of the support surface and one roller closer to the foot of the support surface, wherein the longitudinally-scrolling loop of padding loops around the two rollers and scrolls longitudinally between the two rollers; and a plurality of actuators; and using at least one actuator to lift the flexible support surface up off the loop of padding; (b) using at least one actuator to scroll the loop of padding longitudinally, thereby changing which portion of the loop of padding is directly underneath a selected location on the person's body; and then (c) using at least one actuator to lower the flexible support surface back down onto the loop of padding.

In an example, a device to prevent pressure ulcers can comprise: a flexible support surface (such as a flexible net, mesh, sling, hammock, pad, sheet, or blanket) on which a person lies; a laterally-scrolling loop of padding (such as a loop of compressible foam or a loop of compressible textile padding) which is under the upper support structure; two rollers, one roller closer to the right side of the support surface and one roller closer to the left side of the support surface, wherein the laterally-scrolling loop of padding loops around the two rollers and scrolls laterally between the two rollers; and a plurality of actuators; and using at least one actuator to lift the flexible support surface up off the loop of padding; (b) using at least one actuator to scroll the loop of padding laterally, thereby changing which portion of the loop of padding is directly underneath a selected location on the person's body; and then (c) using at least one actuator to lower the flexible support surface back down onto the loop of padding.

In an example, a device to prevent pressure ulcers can comprise: a flexible upper support surface on which a sleeping person lies; a flexible middle support surface below the upper support surface, wherein the middle support surface is automatically moved along a first axis by one or more actuators, wherein the middle support surface has a first surface portion with a first elasticity level and a second surface portion with a second elasticity level, wherein the second elasticity level is greater than the first elasticity level, and wherein the second surface portion is smaller than the first surface portion; and a flexible lower support surface below the middle support surface, wherein the lower support surface is automatically moved along a second axis by one or more actuators, wherein the second axis is perpendicular to the first axis, wherein the middle support surface has a third surface portion with a third elasticity level and a fourth surface portion with a fourth elasticity level, wherein the fourth elasticity level is greater than the third elasticity level, wherein the fourth surface portion is smaller than the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: a flexible upper support surface on which a sleeping person lies; a flexible middle support surface below the upper support surface, wherein the middle support surface is automatically moved by one or more actuators, wherein the middle support surface has a first surface portion with a first elasticity level and a second surface portion with a second elasticity level, wherein the second elasticity level is greater than the first elasticity level, and wherein the second surface portion is smaller than the first surface portion; and a flexible lower support surface below the middle support surface, wherein the lower support surface is automatically moved by one or more actuators, wherein the middle support surface has a third surface portion with a third elasticity level and a fourth surface portion with a fourth elasticity level, wherein the fourth elasticity level is greater than the third elasticity level, wherein the fourth surface portion is smaller than the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: a plurality of longitudinal cords; a plurality of lateral cords; wherein the longitudinal cords and the lateral cords together create an orthogonal mesh or grid which supports a sleeping person's body; and a plurality of actuators which move (selected) longitudinal cords in a lateral manner and move (selected) lateral cords in a longitudinal manner in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of longitudinal fabric strips; a plurality of lateral fabric strips; wherein the longitudinal fabric strips and the lateral fabric strips together create an orthogonal mesh or grid which supports a sleeping person's body; and a plurality of actuators which move (selected) longitudinal fabric strips apart from each other and move (selected) lateral fabric strips apart from each other in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of longitudinal flexible strips; a plurality of lateral flexible strips; wherein the longitudinal flexible strips and the lateral flexible strips together form an orthogonal mesh or grid which supports a sleeping person's body; and a plurality of actuators which move selected longitudinal strips apart from each other and move selected lateral strips apart from each other in order to enlarge a quadrilateral-shaped opening in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of longitudinal rods; a plurality of lateral rods; wherein the longitudinal rods and the lateral rods together create an orthogonal mesh or grid which supports a sleeping person's body; and a plurality of actuators which move (selected) longitudinal rods apart from each other and move (selected) lateral rods apart from each other in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of longitudinal strips; a plurality of lateral strips; wherein the longitudinal strips and the lateral strips together create an orthogonal mesh or grid which supports a sleeping person's body; and a plurality of actuators which move (selected) longitudinal strips apart from each other and move (selected) lateral strips apart from each other in order to create a quadrilateral-shaped opening in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of longitudinal strips; a plurality of lateral strips; wherein the longitudinal strips and the lateral strips are woven together create an orthogonal mesh or grid which supports a sleeping person's body; and a plurality of actuators which move (selected) longitudinal strips away from each other and move (selected) lateral strips away from each other in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of longitudinal strips; a plurality of lateral strips; wherein the longitudinal strips and the lateral strips are woven together create an orthogonal mesh or grid which supports a sleeping person's body; and a plurality of actuators which move (selected) longitudinal strips apart and move (selected) lateral strips apart in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of porous longitudinal straps; a plurality of porous lateral straps; wherein the longitudinal straps and the lateral straps together create an orthogonal porous mesh or grid which supports a sleeping person's body; and a plurality of actuators which move (selected) longitudinal straps in a lateral manner and move (selected) lateral straps in a longitudinal manner in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of porous longitudinal strips; a plurality of porous lateral strips; wherein the longitudinal strips and the lateral strips together create an orthogonal porous mesh or grid which supports a sleeping person's body; and a plurality of actuators which move (selected) longitudinal strips to overlap each other and move (selected) lateral strips to overlap each other in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of porous longitudinal strips; a plurality of porous lateral strips; wherein the longitudinal strips and the lateral strips together create an orthogonal porous mesh or grid which supports a sleeping person's body; and a plurality of actuators which move (selected) longitudinal strips away from each other and move (selected) lateral strips away from each other in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of porous longitudinal strips; a plurality of porous lateral strips; wherein the longitudinal strips and the lateral strips together create an orthogonal porous mesh or grid which supports a sleeping person's body; and a plurality of actuators which move (selected) longitudinal strips in a lateral manner and move (selected) lateral strips in a longitudinal manner in order to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: a plurality of porous longitudinal strips; a plurality of porous lateral strips; wherein the longitudinal strips and the lateral strips together create an orthogonal porous mesh or grid which supports a sleeping person's body; and a plurality of actuators which move (selected) longitudinal strips and move (selected) lateral strips to create an opening (or hole) in the mesh or grid at a selected location; wherein the selected location is located under a portion of the sleeping person's body which has a pressure ulcer or is particularly vulnerable to getting a pressure ulcer.

In an example, a device to prevent pressure ulcers can comprise: an upper support surface on which a sleeping person lies; a middle support surface below the upper support surface, wherein the middle support surface has a first surface portion with a first elasticity level and a second surface portion with a second elasticity level, wherein the second elasticity level is greater than the first elasticity level, and wherein the second surface portion is smaller than the first surface portion; and a lower support surface below the middle support surface, wherein the middle support surface has a third surface portion with a third elasticity level and a fourth surface portion with a fourth elasticity level, wherein the fourth elasticity level is greater than the third elasticity level, wherein the fourth surface portion is smaller than the third surface portion, and wherein middle support surface and the lower support surface are moved so as to align the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: an upper support surface on which a sleeping person lies; a middle support surface below the upper support surface, wherein the middle support surface is automatically moved by one or more actuators, wherein the middle support surface has a first surface portion with a first elasticity level and a second surface portion with a second elasticity level, wherein the second elasticity level is greater than the first elasticity level, and wherein the second surface portion is smaller than the first surface portion; and a lower support surface below the middle support surface, wherein the lower support surface is automatically moved by one or more actuators, wherein the middle support surface has a third surface portion with a third elasticity level and a fourth surface portion with a fourth elasticity level, wherein the fourth elasticity level is greater than the third elasticity level, wherein the fourth surface portion is smaller than the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

In an example, a device to prevent pressure ulcers can comprise: an upper support surface on which a sleeping person lies; a middle support surface below the upper support surface, wherein the middle support surface has a first surface portion with a first elasticity level and a second surface portion with a second elasticity level, wherein the second elasticity level is greater than the first elasticity level, and wherein the second surface portion is smaller than the first surface portion; and a lower support surface below the middle support surface, wherein the middle support surface has a third surface portion with a third elasticity level and a fourth surface portion with a fourth elasticity level, wherein the fourth elasticity level is greater than the third elasticity level, wherein the fourth surface portion is smaller than the third surface portion, and wherein middle support surface and the lower support surface are moved so as to overlap the second surface portion and the fourth surface portion, thereby creating a localized surface portion with greater elasticity below a selected part of the sleeping person's body that has a pressure ulcer and/or is particularly vulnerable to developing a pressure ulcer.

I claim:

1. A smart bed or mattress for reducing pressure ulcers comprising:
a bed or mattress on which a person rests;
at least one electromagnetic motor;
a first set of flexible straps which span the bed or mattress in a head-to-foot direction; wherein a first subset of straps comprises one or more straps selected from the first set; wherein straps in the first subset have a taut configuration with a first length spanning the bed or mattress and a slack configuration with a second length spanning the bed or mattress; wherein the second length is greater than the first length; wherein straps in the first subset are moved from the taut configurations to the slack configurations, or vice versa, by one or more motors selected from the at least one motor; wherein different first subsets are selected at different times to create low-pressure areas with different sizes and/or locations on the bed or mattress; and wherein the selection of straps in the first subset is based at least partly on predetermined timing, a position of the person's body, analysis of camera images, data from infrared sensors, data from stretch sensors, and/or data from pressure sensors; and
a second set of flexible straps which span the bed or mattress in a right-to-left direction; wherein a second subset of straps comprises one or more straps selected from the second set; wherein straps in the second subset have a taut configuration with a third length spanning the bed or mattress and a slack configuration with a fourth length spanning the bed or mattress; wherein the fourth length is greater than the third length; wherein straps in the second subset are moved from the taut configurations to the slack configurations, or vice versa, by one or more motors selected from the at least one motor; wherein different second subsets are selected at different times to create low-pressure areas with different sizes and/or locations on the bed or mattress; wherein the selection of straps in the second subset is based at least partly on predetermined timing, a position of the person's body, analysis of camera images, data from infrared sensors, data from stretch sensors, and/or data from pressure sensors; wherein a low-pressure area is an area on the bed or mattress where pressure exerted by the bed or mattress on the person's body is reduced relative to other areas of the bed or mattress; and wherein a low-pressure area is formed where the first and second subsets overlap when straps in the first and second subsets are in their slack configurations.

2. A smart bed or mattress for reducing pressure ulcers comprising:
a bed or mattress on which a person rests;
at least one electromagnetic motor;
a first set of flexible straps which span the bed or mattress in a head-to-foot direction; a first subset of straps comprising one or more straps selected from the first set; wherein each strap in the first subset has an elastic segment which is more elastic than the rest of the strap; wherein each strap in the first subset is moved by one or more motors so that the elastic segment is moved to a selected location where a low-pressure area is desired; and wherein the selected location is based at least partly on predetermined timing, a position of the person's body, analysis of camera images, data from infrared sensors, data from stretch sensors, and/or data from pressure sensors; and
a second set of flexible straps which span the bed or mattress in a right-to-left direction; a second subset of straps comprising one or more straps selected from the second set; wherein each strap in the second subset has an elastic segment which is more elastic than the rest of the strap; wherein each strap in the second subset is moved by one or more motors so that the elastic segment is moved to a selected location where a low-pressure area is desired; wherein the selected location is based at least partly on predetermined timing, a position of the person's body, analysis of camera images, data from infrared sensors, data from stretch sensors, and/or data from pressure sensors; wherein a low-pressure area is an area on the bed or mattress where pressure exerted by the bed or mattress on the person's body is reduced relative to other areas of the bed or mattress; and wherein a low-pressure area is formed where the first and second subsets overlap after their elastic segments have been moved.

3. A smart bed or mattress for reducing pressure ulcers comprising:
a bed or mattress on which a person rests;
at least one electromagnetic motor;
a first support layer which spans the bed or mattress in a head-to-foot direction, wherein there is a first opening in the first support layer; and
a second support layer which spans the bed or mattress in a right-to-left direction, wherein there is a second opening in the second support layer, wherein the first support layer and the second support layer are selectively moved by the at least one electromagnetic motor so as to align the first opening and the second opening at a selected location on the bed or mattress, thereby creating a low-pressure area at the selected location, and wherein the selected location is selected at least partly based on predetermined timing, a position of the person's body, analysis of camera images, data from infrared sensors, data from stretch sensors, and/or data from pressure sensors.
